(19) United States
(12) Patent Application Publication
(10) Pub. No.: US 2025/0359984 A1
(43) Pub. Date: Nov. 27, 2025

(54) PROSTHETIC HEART VALVE DELIVERY SYSTEM AND METHOD

(71) Applicant: Shifamed Holdings, LLC, Campbell, CA (US)

(72) Inventors: Ryan William BOYD, Santa Cruz, CA (US); Jasper Ellington ADAMEK-BOWERS, San Francisco, CA (US); Jordan SKARO, San Jose, CA (US); Sarah Louise GARCON, Milpitas, CA (US); Noah R. GOLDSMITH, Santa Cruz, CA (US); Keke LEPULU, Menlo Park, CA (US); Cornelius Matthew CROWLEY, San Francisco, CA (US); Mark QUINTO, San Jose, CA (US); Timothy John CONNEELY, Palo Alto, CA (US); Nicholas J. SPINELLI, San Carlos, CA (US); Randall Scott KOPLIN, Middleton, WI (US); Jonathan Nii Mlin TACKIE, San Francisco, CA (US)

(21) Appl. No.: 18/873,271

(22) PCT Filed: Jun. 9, 2023
(86) PCT No.: PCT/US2023/068213

§ 371 Date: Dec. 9, 2024

Related U.S. Application Data
(60) Provisional application No. 63/366,115, filed on Jun. 9, 2022.

(51) Int. Cl. A61F 2/24; A61M 25/00; A61M 25/01

(52) U.S. Cl. CPC A61F 2/2409; A61F 2/2418; A61F 2/2436; A61M 25/0147; A61M 2025/0004; A61M 2025/015

(57) ABSTRACT

Apparatuses and methods for delivering one or more parts of a valve prosthesis into a patient's heart. The apparatuses may include one or more catheters that are operationally coupled to one or more controls for controlling axial movement, rotational movement and/or deflection of the one or more catheters during the valve prosthesis delivery into the heart. The one or more controls may provide gross and fine movement control over multiple degrees of freedom of one or more catheter, thereby providing superior control for a practitioner during the valve prosthesis delivery procedure.

### CROSS REFERENCE TO RELATED APPLICATIONS

This application claims priority to U.S. Provisional Application No. 63/366,115, filed on Jun. 9, 2022, entitled “PROSTHETIC HEART VALVE DELIVERY SYSTEM AND METHOD”, the entirety of which is incorporated herein by reference for all purposes.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Blood flow between heart chambers is regulated by native valves, i.e., the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully close, causing the patient to experience regurgitation.

While medications may be used to treat diseased native valves, the defective valve may need to be repaired or replaced at some point during the patient's lifetime.

### SUMMARY OF THE DISCLOSURE

Described herein are apparatuses (e.g., devices and systems) and methods for delivering one or more parts of a valve prosthesis into a patient's heart. The apparatuses may include one or more catheters that are operationally coupled to one or more controls for controlling axial movement, rotational movement and/or deflection of the one or more catheters. The control(s) may provide gross and fine movement control over multiple degrees of freedom of the catheter(s), thereby providing superior control for a practitioner during the valve prosthesis delivery procedure.

According to some examples, a delivery system for a prosthetic heart valve, the prosthetic heart valve comprising an anchor adapted to be disposed in a ventricle adjacent a native valve of a patient's heart and a frame supporting valve leaflets adapted to be expanded within the anchor, the delivery system comprises: an anchor control catheter adapted to be advanced into an atrium of the patient's heart, the anchor control catheter comprising: a lumen extending from a proximal end to a distal end of the anchor control catheter, the lumen being sized and configured to slidingly contain the anchor; a distal guide arm in a distal portion of the anchor control catheter, at least a portion of the distal guide arm having an at-rest helical or spiral shape; and a proximal controller at the proximal end of the anchor control catheter, the proximal controller being configured to change a shape of the distal guide arm. The distal guide arm may have a proximal portion and a distal portion, the proximal portion comprising the portion of the distal guide arm having an at-rest helical or spiral shape. The proximal controller may comprise an actuator operatively connected to the anchor in the lumen of the anchor control catheter to move the anchor distally and proximally within the lumen to change the shape of the distal portion of the distal guide arm. The actuator may be connected to a tether which is removably connected to the anchor. The proximal controller may comprise an actuator operatively connected to the distal portion of the distal guide arm and adapted to change a shape of the distal portion of the distal guide arm. The actuator may be connected to an actuation catheter movably disposed within the lumen of the anchor control catheter, a distal end of the actuation catheter being connected to the distal portion of the distal guide arm. The proximal controller may comprise an actuator operatively connected to a proximal portion of the anchor control catheter and adapted to rotate the anchor control catheter. The distal guide arm may be sized and configured to move to a spiral shape within the atrium of the patient's heart. The proximal controller may be further configured to extend the distal guide arm from the atrium through valve leaflets into the ventricle with the anchor disposed within the lumen. The proximal controller may be further configured to move a distal end of the distal guide arm within the ventricle to encircle chordae of the heart with the distal guide arm. The proximal controller may further be configured to withdraw the anchor control catheter from the anchor after the distal guide arm has encircled the chordae.

According to another example, a delivery system for a prosthetic heart valve, the prosthetic heart valve comprising an anchor adapted to be disposed in a ventricle adjacent a native valve of a patient's heart and a frame supporting valve leaflets adapted to be expanded within the anchor, the delivery system comprises: a valve capsule, the valve frame being disposed within the valve capsule in a compressed configuration; a capsule shaft catheter connected to the valve capsule and extending proximally from the valve capsule; a valve retainer removably connected to the valve frame; and a proximal controller at a proximal end of the capsule shaft catheter, the proximal controller being configured to remove the capsule from the valve frame, thereby permitting the valve frame to expand. The delivery system may further comprise an inner steerable catheter disposed within a lumen of the capsule shaft catheter and an inner catheter steering control line extending from a distal portion of the inner steerable catheter to the proximal controller, the proximal controller being further configured to apply and release tension on the inner catheter steering control line. The delivery system may further comprise an outer steerable catheter and an outer catheter control line extending from a distal portion of the outer steerable catheter to the proximal controller, the proximal controller being further configured to apply and release tension on the outer catheter control line, the capsule shaft catheter being disposed in a lumen of the outer steerable catheter. The capsule shaft catheter may include multiple axial sections having different stiffnesses, thereby providing different degrees of deflection when activated. When the capsule shaft catheter is in a deflected state, the capsule shaft catheter may include a first bend and a second. The first bend may be configured to be in a right atrium of the patient's heart and the second bend is configured to be within a left atrium of the patient's heart.

According to a further example, a track system is adapted to control movement of a catheter system for delivering at least a portion of a prosthetic heart valve into a patient's heart, wherein the catheter system includes a first catheter coaxially arranged with a second catheter, the track system comprising: a primary track and a secondary track positioned in parallel; a first carriage adapted to secure a proximal portion of the first catheter thereto and to translate along the primary track, wherein the first carriage is coupled to the secondary track such that the secondary track translates with the first carriage when the first carriage translates along the primary track; and a second carriage adapted to secure a proximal portion of the second catheter thereto and to translate along the primary track, wherein the second carriage includes a coupler that is adapted to selectively engage the second carriage with the secondary track such that, when the coupler is engaged, the second carriage translates with the first carriage when the first carriage translates along the primary track. The first carriage may include a fastener that is configured to transition between: a first closed state in which the proximal portion of the first catheter is frictionally secured to the first carriage, wherein the first catheter is maintained at an intended rotational position but is rotatable with respect to the first carriage; and a second closed state in which the proximal portion of the first catheter is fully secured to and not rotatable with respect to the first carriage. The track system may further comprise a third carriage adapted to secure a proximal portion of a third catheter thereto and to translate along the primary track, wherein the third carriage includes a second coupler that is adapted to selectively engage the third carriage with the secondary track such that, when the second coupler is engaged, the third carriage translates with the first carriage when the first carriage translates along the primary track. The first carriage may include a first fastener configured to releasably secure the proximal portion of the first catheter thereto, and the second carriage includes a second fastener configured to releasably secure the proximal portion of the second catheter thereto, wherein each of the first and second fasteners are configured to releasably secure a proximal portion of a different catheter thereto. The coupler may be adapted to disengage the second carriage from the secondary track such that, when the coupler is disengaged, the second carriage translates independently from the first carriage. The coupler may be disengaged in a default state. The track system may further comprise a rail that supports the primary and secondary tracks in parallel. The first carriage may include a first gear assembly adapted to translate the first carriage along the primary track, and wherein the second carriage includes a second gear assembly adapted to translate the second carriage along the primary track. The first catheter may be slidably positioned within the second catheter. The second catheter may be slidably positioned within the first catheter. The second carriage may include a button adapted to engage and disengage the coupler. Each of the first and second carriages may include a gear assembly that is configured to engage with teeth of the primary track when the respective first or second carriage translates along the primary track. Each of the first and second carriages may include a dial that is configured to translate the respective first or second carriage along the primary track upon rotation of the dial. Each of the first and second carriages may comprise a lock to lock a translational position of the first or second catheter relative to the primary track.

According to another example, a method of delivering an anchor of a prosthetic heart valve into a patient's heart, the method comprises: advancing an anchor control catheter into an atrium of the patient's heart, the anchor control catheter having a distal guide arm, wherein the anchor is slidably positioned within the anchor control catheter; advancing the guide arm through a native valve annulus and into a ventricle of the patient's heart, wherein the guide arm has a first shape and a distal end; and rotating the guide arm to capture chordae near the native valve annulus with the distal end of the guide arm, wherein capturing the chordae comprises moving the anchor within the guide arm such that the anchor applies a force against the guide arm to change the first shape of the guide arm to a second shape and to change a distance to which the distal end of the guide arm radially extends. Changing the first shape of the guide arm to the second shape may comprise changing a radius of curvature of the distal end of the guide arm. The anchor control catheter may be positioned with a steerable catheter having a deflected configuration when the guide arm is capturing the chordae, wherein capturing the chordae further comprises adjusting the steerable catheter to alter a position of the guide arm within the ventricle. The guide arm may comprise a proximal end extending generally along a first axis, and wherein the distal end of the guide arm is in a plane that is substantially perpendicular to the first axis, and further wherein the change in distance is with respect to the first axis. Each of the first and second shapes of the guide arm may have a helical shape or a spiral shape.

According to an additional example, a delivery system for delivering an anchor of a prosthetic heart valve into a patient's heart comprises: a catheter assembly having the anchor slidably positioned within an anchor control catheter, wherein the anchor control catheter is slidably positioned within a steerable catheter, wherein a distal portion of the anchor control catheter includes a guide arm with a distal end; and a controller coupled to a proximal portion of the anchor control catheter, wherein the controller comprises: a first control configured to apply a pre-load force the guide arm while the guide arm is within the steerable catheter such that the guide arm self-assembles into a spiral or helical shape when the guide arm is advanced out of the steerable catheter; and a second control configured to move the anchor within the guide arm to apply force against the guide arm that changes a distance to which the distal end of the guide arm radially extends. The controller may further comprise a third control configured to control an axial height of the guide arm relative to the steerable catheter. The third control may be part of a carriage that is releasably coupled to the proximal portion of the anchor control catheter, wherein the third controller is configured to translate the proximal portion of the anchor control catheter on a rail relative to a proximal portion of the steerable catheter.

According to a further example, a system for controlling movement of a catheter for delivering at least a portion of a prosthetic heart valve into a patient's heart comprises: a handle coupled to a proximal portion of the catheter, the handle comprising a control configured to control deflection of a distal portion of the catheter; and a carriage including a fastener that is configured to secure the handle to a support, the fastener including a band that is configured to surround the handle to secure the handle to a cradle, wherein the fastener is configured to transition among: an open state in which the band is in an open position such that the handle can be removed from the cradle; a first closed state in which the band loosely surrounds the handle, and the handle is frictionally secured to the cradle at an intended rotational position but is rotatable with respect to the carriage; and a second closed state in which the band securely surrounds the handle such that the handle is rotatably fixed with respect to the carriage. The support may include a track system that is configured to allow translation of the carriage with the handle fastened thereto to allow axial movement of the distal portion of the catheter. The handle may be a first handle coupled to a first catheter, and the carriage may be a first carriage, wherein the system may further comprise: a second handle coupled to a proximal portion of a second catheter that is coaxially aligned with the first catheter; and a second carriage that is configured to secure the second handle to the track system, wherein the first and second carriages are configured to independently translate along the track system to cause independent axial movement of the distal portions of the first and second catheters. The track system may be configured to selectively allow coupled translation of the first and second carriages together along the track system to cause coupled axial movement of the distal portions of the first and second catheters. The cradle may include one or more engagement features that is configured to frictionally engage with corresponding features of the handle to maintain the in handle in the intended rotational position.

According to an additional example, a delivery system adapted to deliver an anchor of a prosthetic heart valve into a patient's heart comprises: an anchor control catheter having a distal guide arm that is configured to take on a spiral or helical shape, wherein the anchor is slidably positioned within the anchor control catheter; and a handle coupled to a proximal portion of the anchor control catheter, wherein the handle includes: a first control that is configured to bias the distal guide arm toward the spiral or helical shape; and a second control that is configured to axially move the anchor within the anchor control catheter to change an extent to which a distal end of the distal guide arm radially extends. The second control may be configured to radially extend the distal end of the distal guide arm to capture chordae of the patient's heart, thereby allowing encircling of the distal guide arm around the chordae. The delivery system may further comprise a steerable catheter in which the anchor control catheter is slidably positioned within, where the first control is configured to bias the distal guide arm toward the spiral or helical shape while the distal guide arm is within the steerable catheter. The delivery system may further comprise a second handle coupled to the steerable catheter, wherein the second handle includes a deflection control that is configured to selectively deflect a distal portion of the steerable catheter to steer the distal guide arm within the patient's heart. The delivery system may further comprise a second handle coupled to the steerable catheter, wherein the second handle is translatable with respect to the first handle to axially retract a distal portion of the steerable catheter with respect to the distal guide arm to allow the distal guide arm to be released from the steerable catheter and take on the spiral or helical shape. The delivery system may further comprise a rail system comprising a first carriage configured to fasten the first handle to the rail system and a second carriage configured to fasten the second handle to the rail system, wherein the first and second carriages are translatable along a track.

According to another example, a method of delivering an anchor of a prosthetic heart valve into a patient's heart comprises: advancing a catheter system into the atrium of the patient's heart, wherein the catheter system includes an anchor control catheter positioned within a steerable catheter, wherein the anchor is positioned within the anchor control catheter, and wherein the anchor control catheter includes a distal guide arm; biasing the distal guide arm toward a spiral or helical shape while the distal guide arm is within the steerable catheter; and advancing the distal guide arm such that the distal guide arm exits a distal end of the steerable catheter and takes on the spiral or helical shape. Biasing the distal guide arm may comprise activating a control of a handle coupled to a proximal portion of the anchor control catheter. The method may further comprise advancing the distal guide arm through a native valve annulus by translating the handle along a rail system. The method may further comprise encircling chordae near the native valve annulus with the distal guide arm, wherein encircling the chordae comprises changing an extent to which a distal end of the guide arm radially extends by axially moving the anchor within the distal guide arm. The method may further comprise retracting the distal guide arm over the anchor to release the anchor from the distal guide arm, wherein retracting the distal guide arm comprises translating the handle along the rail system.

According to an additional example, a delivery system adapted to deliver a prosthetic heart valve into a patient's heart comprises: a steerable catheter having a distal valve capsule configured to hold a frame of the prosthetic valve therein; and a handle coupled to a proximal portion of the steerable catheter, wherein the handle includes: a valve deployment knob that is configured to control retraction the distal valve capsule with respect to the frame to release at least a portion of the frame from the steerable catheter; a depth control knob that is configured to control axial movement of the distal portion of the steerable catheter; and a deflection knob that is configured to control deflection of the distal portion of the steerable catheter. The handle may be translatably coupled to a track system, wherein the track system includes a translation control that is configured to translate the handle to control gross axial movement of the distal portion of the steerable catheter. The steerable catheter may include multiple axial sections having different degrees of flexibility, wherein deflection of the steerable catheter causes the distal portion of the steerable catheter have a first bend and a second bend separated by a reach section of the steerable catheter.

According to a further example, a method of delivering ca prosthetic heart valve into a patient's heart comprises: advancing a steerable catheter over a guide wire into an atrium of the patient's heart, the steerable catheter having a proximal portion coupled to a handle and a distal portion having a valve capsule holding a frame of the prosthetic heart valve therein, wherein advancing the steerable catheter into the atrium comprises translating the handle with respect to a support translatably coupled to the handle; steering the valve capsule toward a native valve annulus of the patient's heart by deflecting the steerable catheter, wherein the deflecting comprises activating a deflection knob of the handle; advancing the valve capsule partially through the native valve annulus of the patient's heart by activating a depth control knob of the handle; and releasing the frame of the prosthetic heart valve into the native valve annulus by activating a valve deployment knob of the handle that retracts the valve capsule with respect to the frame, wherein the frame expands into the native valve annulus and within an anchor that encircles chordae near the native valve annulus. The method may further comprise: releasing a ventricle side of the frame within the ventricle of the patient's heart by activating the valve deployment knob of the handle; and pulling the ventricle side of the frame toward the native valve annulus to position the anchor closer to the native valve annulus by activating the depth control knob. The steerable catheter may be in a deflected state when pulling the ventricle side of the frame toward the native valve annulus, wherein the steerable catheter includes a first bend within a right atrium of the patient's heart and a second bend within a left atrium of the patient's heart. The support may include a rail system, wherein the handle is coupled to the rail system by a carriage that is translatably coupled to a track, wherein translating the handle comprises activating a dial of the carriage to translate the carriage with respect to the track.

According to another example, a method of delivering a prosthetic heart valve into a patient's heart comprises: advancing a steerable catheter over a guide wire into an atrium of the patient's heart, the steerable catheter having a proximal portion coupled to a handle and a distal portion having a valve capsule holding a frame of the prosthetic heart valve therein, wherein advancing the steerable catheter into the atrium comprises translating the handle with respect to a support translatably coupled to the handle; advancing the valve capsule partially through a native valve annulus of the patient's heart by activating a depth control knob of the handle, wherein an anchor of the prosthetic heart valve encircles chordae near the native valve annulus; releasing a ventricle side of the frame within a ventricle of the patient's heart by activating a valve deployment knob of the handle; pulling the ventricle side of the frame toward the native valve annulus to position the anchor closer to the native valve annulus by activating the depth control knob of the handle; and releasing an atrium side of the frame within the atrium of the patient's heart by activating the valve deployment knob of the handle to fully retract the valve capsule with respect to the frame, wherein the frame expands into the native valve annulus and within the anchor. The anchor may be freely implanted within the patient's heart while the ventricle side of the frame is pulled toward the native valve annulus. The anchor may not be coupled to a tether. The method may further comprise steering the valve capsule toward the native valve annulus by deflecting the steerable catheter, wherein the deflecting comprises activating a deflection knob of the handle. The steerable catheter may be in a deflected state when pulling the ventricle side of the frame toward the native valve annulus, wherein the steerable catheter includes a first bend within a right atrium of the patient's heart and a second bend within a left atrium of the patient's heart. The support may include a rail system, wherein the handle is coupled to the rail system by a carriage that is translatably coupled to a track, wherein translating the handle comprises activating a dial of the carriage to translate the carriage with respect to the track.

According to an additional example, a method of delivering a prosthetic heart valve into a patient's heart comprises: advancing an anchor delivery catheter system into the patient's heart, wherein the anchor delivery catheter system includes an anchor slidably positioned within an anchor control catheter, and the anchor control catheter is slidably positioned within a steerable catheter, wherein a distal portion of the anchor control catheter includes a guide arm, wherein a proximal portion of the steerable catheter is coupled to a first handle and a proximal portion of the anchor delivery catheter is coupled to a second handle, wherein the first and second handles are translatably coupled to a rail system; implanting the anchor around chordae near a native valve of the patient's heart, wherein implanting the anchor comprises translating the first handle along the rail system independent of the second handle; removing the anchor delivery catheter system from the rail system and coupling a valve delivery catheter system to the rail system, wherein a steerable catheter handle of the valve delivery catheter system is translatably coupled to the rail system, wherein the valve delivery catheter system includes a frame of the prosthetic heart valve therein; and advancing the valve delivery catheter into the patient's heart and deploying the frame into the native valve of the patient's heart and within the implanted anchor, wherein advancing the valve delivery catheter comprise translating the steerable catheter handle along the rail system. The first handle may be releasably coupled to a first carriage that is translatably coupled to the rail system, and wherein the second handle is releasably coupled to a second carriage that is translatably coupled to the rail system. Translating the first handle along the rail system may comprise translating the first carriage independent of the second carriage. The steerable catheter handle may be coupled to the first carriage or the second carriage. Implanting the anchor may further comprise unlocking a fastener that secures the second handle to the rail system, and rotating the second handle to rotate a guide arm at a distal end of the anchor control catheter, wherein rotating the guide arm comprise capturing chordae within the guide arm.

These and other examples are described herein.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates an example schematic prosthetic mitral valve in place in a patient's heart.

FIG. 2 illustrates an example anchor delivery subsystem.

FIG. 3 illustrates an example section view of a nested catheter for the anchor delivery subsystem of FIG. 2.

FIG. 4A illustrates an example section view of an inner steerable catheter.

FIG. 4B illustrates an example section view of an outer steerable catheter.

FIG. 5 illustrates an example perspective view of a coil layer.

FIG. 6 illustrates an example perspective view of a braid layer.

FIG. 7 illustrates an example perspective view of another braid layer.

FIGS. 8A and 8B illustrate example perspective and front views, respectively, of a pull ring.

FIG. 9 illustrates an example perspective view of a jacket assembly.

FIGS. 10 and 11 illustrate example perspective views of a capstan assembly.

FIGS. 12A-12B illustrate example schematic section views of an anchor control catheter.

FIG. 13A illustrates an example side view of a rotational control shaft.

FIGS. 13B, 13C and 13D illustrate example front views of a laser cut patterns of different regions of the rotation control shaft of FIG. 13A.

FIGS. 14A and 14B illustrate an example of a spiral guide arm.

FIGS. 15A and 15B illustrate an example of a helical guide arm.

FIG. 16A illustrates a sample cross-sectional image of the spiral guide arm of FIGS. 14A and 14B.

FIG. 16B illustrates a sample cross-sectional image of the helical guide arm of FIGS. 15A and 15B.

FIGS. 17A-17D illustrate example support structures for intermediate parts of guide arms.

FIG. 18A illustrates an example guide arm in a self-assembly position in which the anchor within the guide arm is fully axially extended.

FIGS. 18B and 18C illustrate the guide arm of FIG. 18A in an encircling position in which the anchor is retracted within the guide arm.

FIGS. 19A-19D illustrate details of an example anchor.

FIG. 20 illustrates an example of part of a proximal controller for an anchor control catheter and tether.

FIG. 21 illustrates an example of part of another proximal controller that combines into one handle actuators for an anchor control catheter and tether.

FIG. 22 illustrates an example proximal controller for an anchor delivery subsystem.

FIG. 23 illustrates an example top view of an anchor.

FIG. 24 illustrates an example valve delivery subsystem.

FIG. 25A illustrates an example section view of a nested catheter for the valve delivery subsystem of FIG. 24.

FIG. 25B illustrates an example distal portion of a valve delivery subsystem.

FIG. 26A illustrates an example tab retainer as part of the valve delivery subsystem.

FIG. 26B illustrates an example tab retainer shaft as part of the valve delivery subsystem.

FIG. 27A-27C illustrate an example steerable catheter that may be used as a single steerable catheter as part of a valve delivery subsystem.

FIG. 28 illustrates another example view of a proximal controller for the valve delivery subsystem.

FIGS. 29A and 29B illustrate examples of a valve prosthesis having a valve frame structure.

FIGS. 30A and 30B illustrate another example proximal controller for an anchor delivery subsystem.

FIG. 31 illustrates an exploded view of an example proximal controller for an anchor delivery subsystem, such as the anchor delivery subsystem of FIGS. 30A and 30B.

FIG. 32 illustrates another example proximal controller for a valve delivery subsystem.

FIGS. 33A to 33V and 33-1 to 33-13 illustrate systems and methods for implanting an anchor and a prosthetic mitral valve in a heart of a subject.

### DETAILED DESCRIPTION

This disclosure is directed to a delivery system for a prosthetic heart valve that has two main components: an anchor adapted to be disposed in a ventricle adjacent a native valve of a patient's heart and a frame supporting prosthetic valve leaflets adapted to be delivered after delivery of the anchor and then expanded within the anchor. In particular, the valve is a prosthetic mitral valve, and the delivery system of this invention delivers the valve's two components transeptally. In use, the delivery system advances distally from an entry point in the patient's femoral vein, enters the right atrium of the heart, and passes through the septum into the left atrium to implant the anchor and then expand the valve frame inside the anchor.

Because the anatomy of the heart may differ from patient to patient, it may be desirable to be able to control the movement, position, and/or orientation of the delivery system while delivering and implanting the anchor and the valve frame. It may also be necessary to retrieve the anchor and/or the valve during implantation if their position is not quite right. The prosthetic valve delivery system of this invention therefore provides mechanisms for navigating the anchor and the valve and for controllably releasing the anchor and the valve when they have been correctly placed.

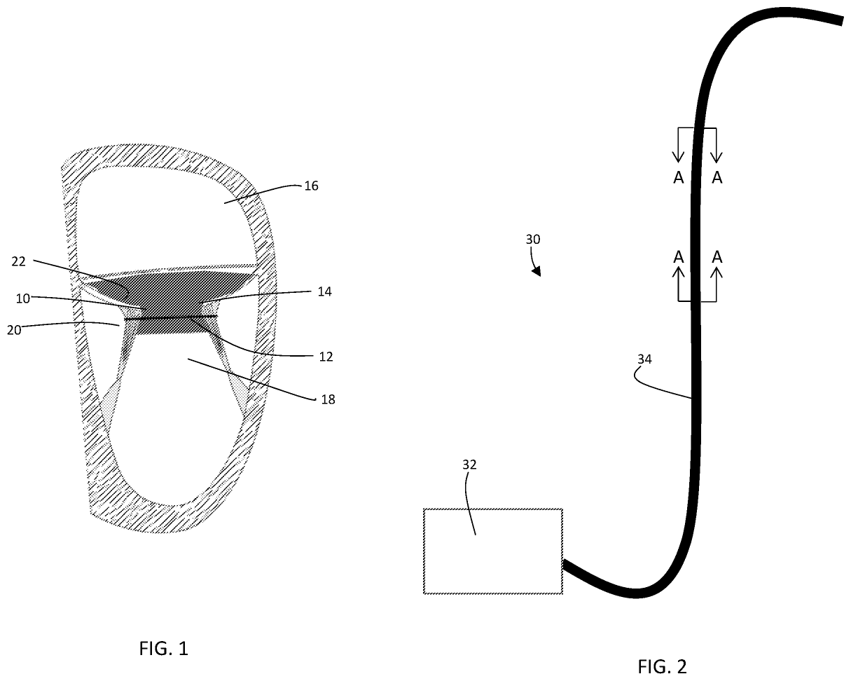

FIG. 1 shows an exemplary prosthetic mitral valve 10 in place in a patient's heart. Valve 10 includes an anchor 12 and a valve frame 14. Moveable leaflets (not shown) attached to the valve frame take the place, and perform the function, of the native valve leaflets. As shown, anchor 12 has been placed around chordae tendineae 20 in the left ventricle 18. Valve frame 14 extends between the left atrium 16 and the left ventricle 18 through the native valve annulus 22.

The anchor 12 and valve frame 14 of valve 10 are implanted separately. Anchor 12 is delivered first and placed around the chordae 20. Valve frame 14 is thereafter delivered and expanded within anchor 12. In order to advance the valve components from an opening in the patient's groin to the heart, the delivery system might need to be pushed, bent, and/or rotated to navigate the anatomy of the intervening vasculature.

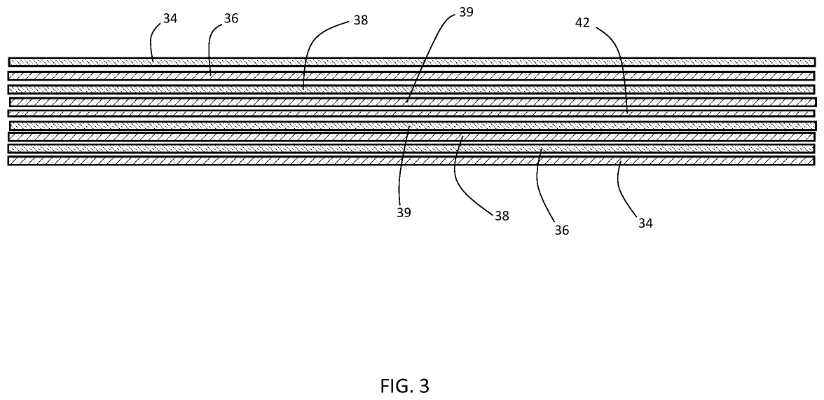

Because the anchor and the frame are delivered separately, the delivery system described herein has two main subsystems: An anchor delivery subsystem and a valve frame delivery subsystem. FIGS. 2 and 3 show aspects of an anchor delivery subsystem 30 having a proximal controller 32 and three nested catheters (as shown in cross-section in FIG. 3): An outer steering catheter 34, an inner steering catheter 36 movably disposed within the lumen of the outer steering catheter 34, and an anchor control catheter 38 with an outer rotation shaft 40 and an inner actuation catheter 39 movably disposed within the lumen of the inner steering catheter. A guide arm (not shown in FIG. 3) extends from a distal end of the outer rotation shaft 40 of the anchor control catheter 34, as described below. Also shown in FIG. 3 is a tether 42 releasably connected at its distal end to an anchor (not shown) movably disposed within the inner steering catheter 36. Outer steering catheter 34, inner steering catheter 36, anchor control catheter 38, and tether 42 are all operatively connected to the proximal controller 32. An introducer sheath (not shown) may be used to introduce the three nested catheters into the patient's vasculature.

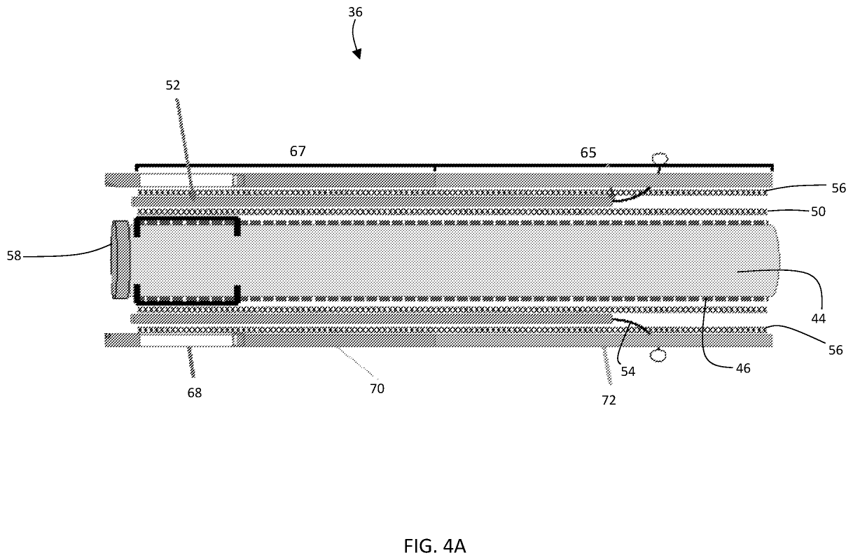

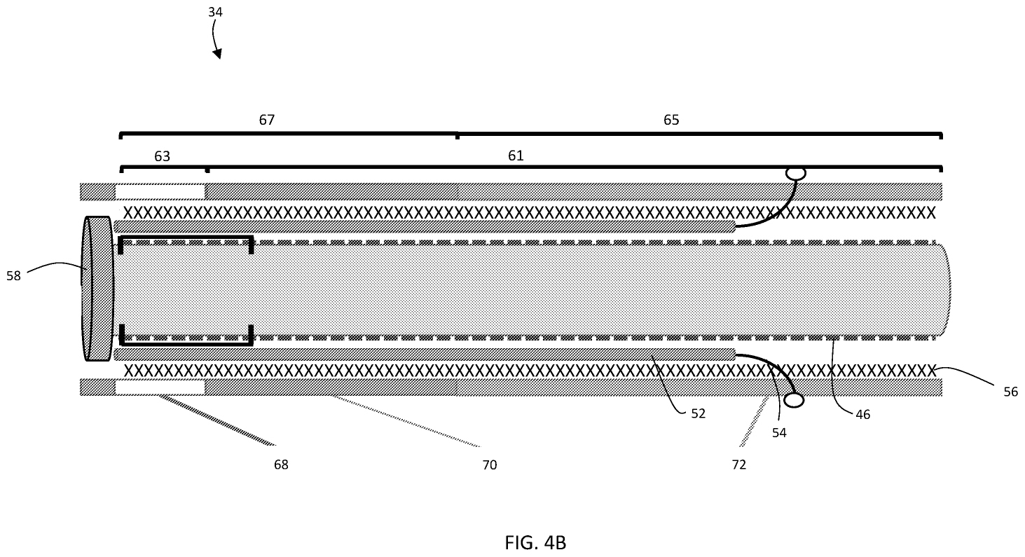

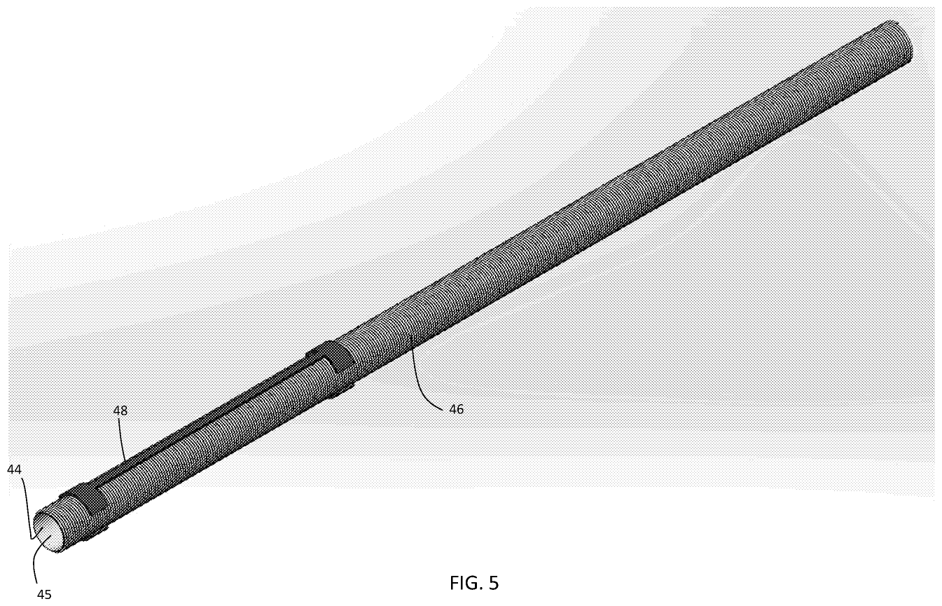

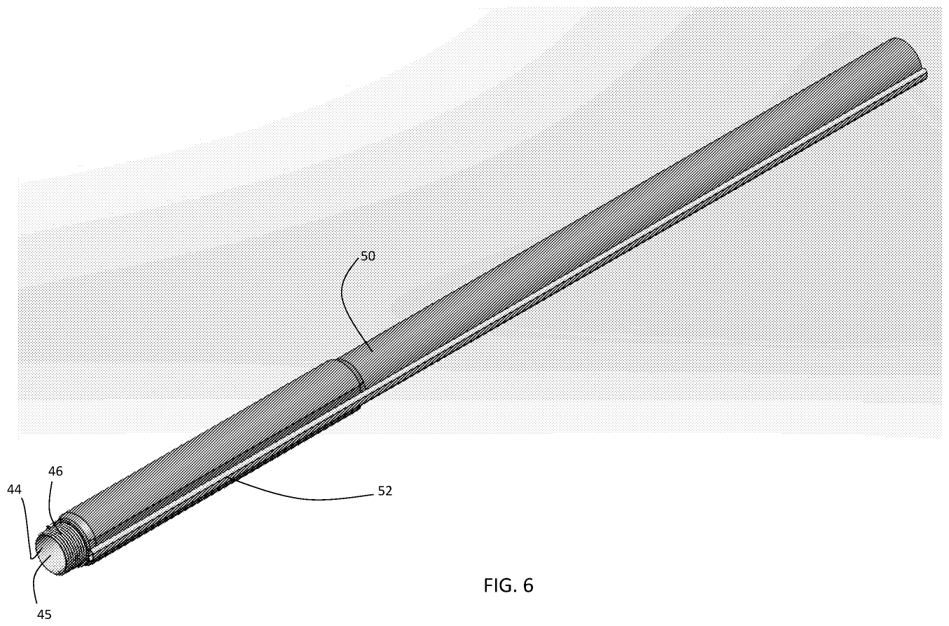

FIGS. 4A and 6 show aspects of a catheter that can be employed as the inner steerable catheter 36, and FIG. 4B shows aspects of a catheter than can be employed as the outer steerable catheter 34. FIGS. 5 and 7-11 show features common to both the inner steerable catheter and the outer steerable catheter with reference to their use with the inner steerable catheter 36.

Each of the steerable catheters 34 and 36 has a liner 44 formed from, e.g., PTFE or other suitable material surrounding a lumen 45. A first coil layer 46 surrounds the liner. In the inner steerable catheter 36, as shown in FIG. 4B, the coil layer 46 may be formed, for example, using wire at a first pitch, while the coil layer 46 of outer steerable catheter 34 may be formed of wire at the first pitch in a proximal region 61 and a second pitch (e.g., larger pitch) in a distal region 63. Two axial reinforcement members 48 may be placed 180° apart over coil layer 46; one axial reinforcement member 48 is shown in FIG. 5.

In the inner steerable catheter 36, as shown in FIGS. 4A and 6, an inner braid layer 50 surrounds the coil layer 46 and reinforcement member(s) 48. Braid layer 50 may be, e.g., a diameter double ended wire braid. Two longitudinal pull line lumens 52 disposed 180° apart are placed over braid layer 50, each at locations 90° offset from the axial reinforcement members 48. (Only one pull line lumen is shown in FIG. 6.) Pull line lumens 52 may be formed from, e.g., PTFE. Pull lines 54 (formed, e.g., from Vectran® fibers) extend through the lumens 52, as explained further below.

In both the inner steerable catheter 36 and outer steerable catheter 34, a braid layer 56 extends around pull line lumens 52. Braid layer 56 may be, e.g., a braid in the inner steerable catheter 36 and a double ended braid in the outer steerable catheter 34. In the inner steerable catheter 36 and the outer steerable catheter 34, the braid 56 may have a first braid density (ppi) at a proximal region 65 and a second braid density (ppi) (e.g., greater than the first braid density) at a distal region 67. A pull ring 58 is disposed over braid layer 56 at the distal end of the catheter.

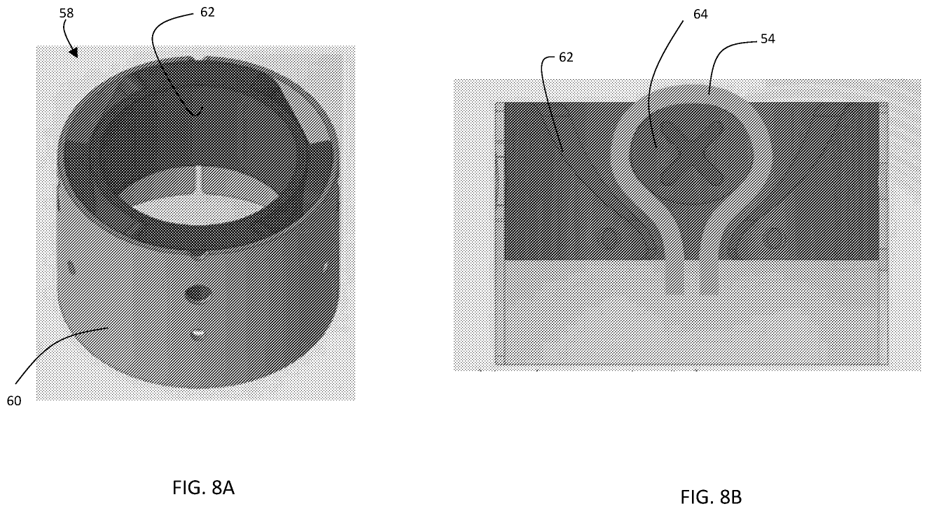

As shown in FIGS. 8A-8B, pull ring 58 may be formed with an outer ring 60 welded to an inner ring 62. Pull lines 54 are looped around bollards 64 disposed between the inner and outer rings 180° apart. The free ends of pull lines 54 extend proximally through the pull line lumens 52 to the proximal controller.

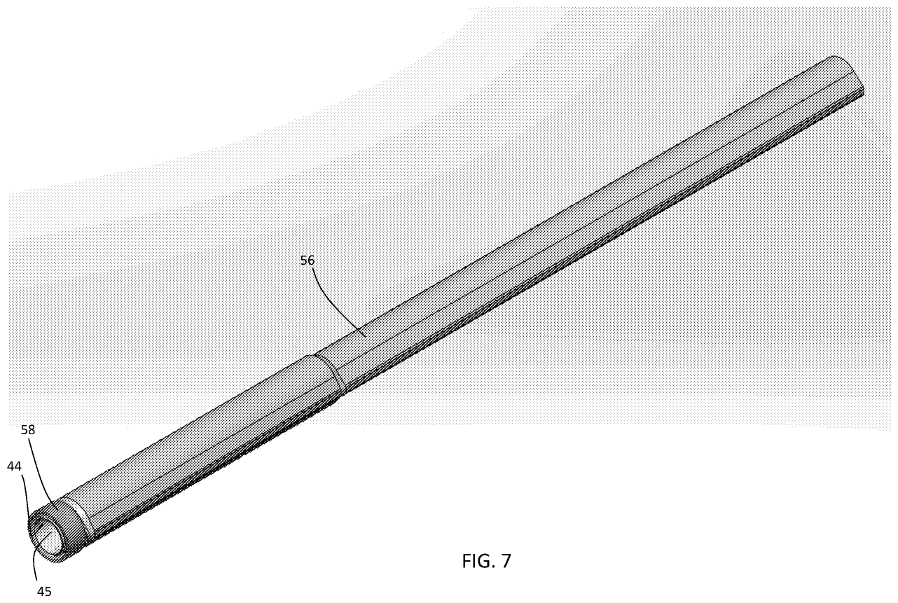

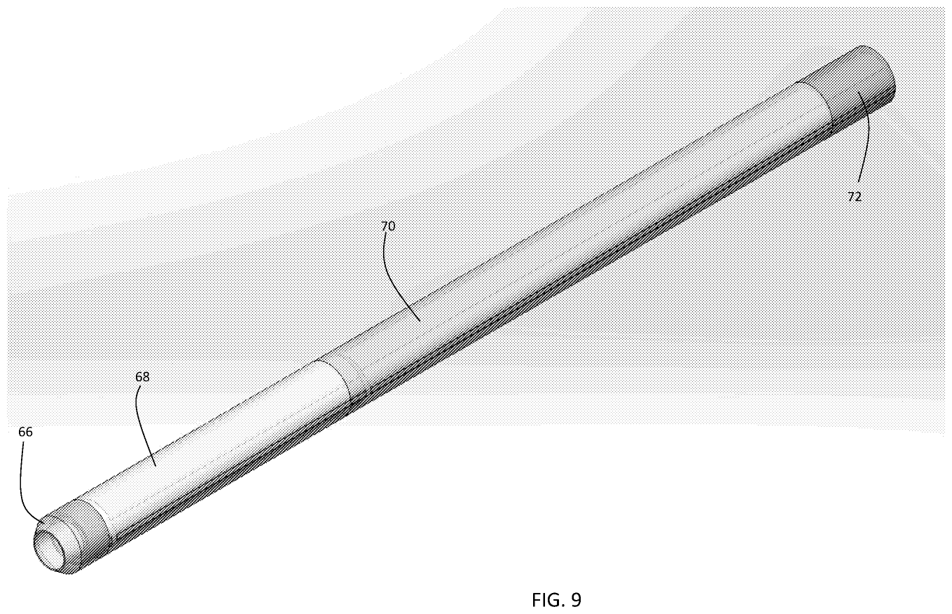

FIG. 9 shows a distal tip 66 that extends around pull ring 58. Distal tip 66 may be formed, e.g., from a polymer (e.g., Pebax®). A distal end of liner 44 (FIG. 7) is everted around distal tip 66. Three outer jackets cover sections of the catheter. A flexible distal outer jacket 68 (formed, e.g., from Tecoflex® or Tecothane® polymer) extends proximally from the distal tip 66. Extending proximally from distal outer jacket 68 is a middle jacket 70 having a flexibility less than that of distal outer jacket 68 (formed from, e.g., a polymer (e.g., Pebax®)) to provide an intermediate level of bending stiffness while provide sufficient stiffness to transmit torque to the distal portion of the catheter. A proximal outer jacket 72 having a stiffness greater than that of the middle jacket 70 (formed, e.g., from Vestamid® polymer) extends proximally from middle jacket 70. The stiffness of proximal outer jacket 72 provides a good torque response and has low compression and elongation characteristics.

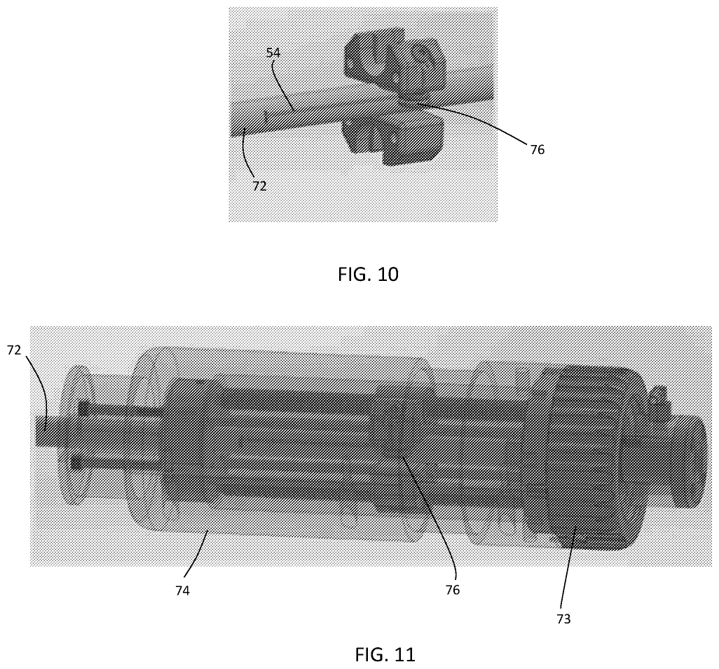

FIGS. 10 and 11 show the free ends of pull lines 54 extend from the proximal ends of pull line lumens 52 (e.g., FIG. 6) through openings in the braid layer 56 and in the proximal outer jacket 72 to a handle 74 within the proximal controller at the proximal end of catheter 34. The free ends of pull lines 54 are wrapped around a pair of capstans 76 disposed within the proximal controller 74. The capstans 76 ride on leadscrews that are actuated by a ring gear attached to rotational knob 73 on the proximal end of the handle. One capstan is connected to a right-handed leadscrew, and the other capstan is connected to a left-handed leadscrew, so that the two capstans 76 rotate in equal amounts in opposite directions when actuated by knob 73 to deflect the distal end of catheter 34.

When used together, the outer and inner steering catheters can be used to navigate the patient's vasculature from their insertion point in the patient's groin through the vasculature to the patient's heart. Each of the inner steerable catheter and the outer steerable catheter may be steered in a single plane. The outer steerable catheter may be used to navigate from the vascular entry point in the femoral vein through the vena cava to the right atrium and through the septum into the left atrium. The inner steerable catheter may be used to navigate from the septal crossing toward and through the native mitral valve into the left ventricle.

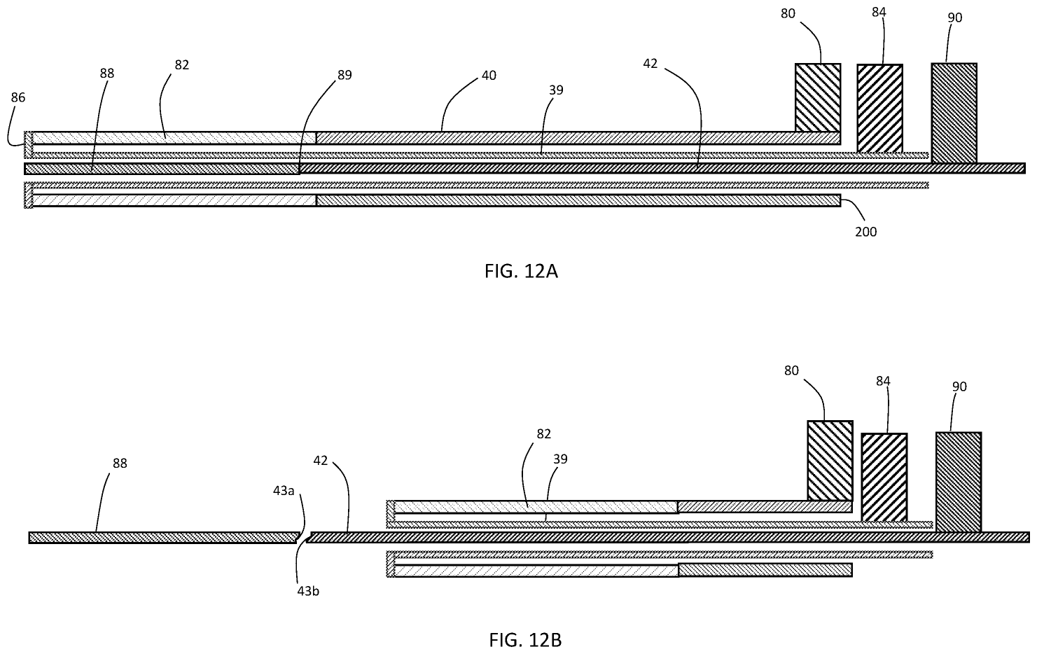

FIGS. 12-19 show aspects of the anchor control catheter 38. After the outer and inner steering catheters have been used to advance the distal end of the inner steering catheter from the patient's right atrium through the septal wall into the left atrium, the anchor control catheter 38 is used to deliver and deploy the anchor of the prosthetic valve. FIG. 12A is a schematic cross-sectional representation of the major components of the anchor control catheter 38. A rotation control shaft 40 of the anchor control catheter 38 extends distally from an actuator 80 in a proximal controller. A guide arm 82 extends distally from the rotation control shaft 40.

Guide arm 82 has active and passive features that enable it to assemble into a spiral in the left atrium, as described below. Specifically, guide arm 82 has shape set features and cut pattern features that enable the guide arm to be flexible when within the steerable catheters. When it emerges from the inner steerable catheter, however, guide arm 82 achieves a desired shape through a combination of the shape set features and actuation of the cut pattern to hold the desired shape.

Actuation catheter 39 extends through the lumen of the rotation control shaft 40 and guide arm 82 from an actuator 84 in the proximal controller to the distal end of the guide arm 82. A cap 86 extends over, and is attached to, the distal end of guide arm 82. Cap 86 is a 72D PEBAX tip that couples the distal end of the actuation catheter 39, distal end of the guide arm 82, and the outer jacket into a smooth and atraumatic distal tip. Proximal movement of actuator 84 causes places guide arm 82 and rotation control shaft 40 in compression to change the functional features of these elements, as described below.

Rotational movement of actuator 80 rotates the rotation control shaft 40, guide arm 82, and actuation catheter 39. Rotation control shaft 40 is a laser-cut hypotube designed to be flexible and to transmit the rotational force along the length of the anchor control catheter 38. Rotation control shaft 40 is configured to effectively transmit torque from actuator 80 to guide arm 82 to ensure that rotation of the proximal end of rotation control shaft 40 results in a substantially equal amount of rotation of guide arm 82 at the distal end of rotation control shaft 40. Each region of rotation control shaft 40 is cut in a pattern (or not cut at all) to provide features useful to that region.

Tether 42 extends from an actuator 90 in the proximal controller to the anchor 88 of the prosthetic heart valve. The tether 42 is releasably attached to or abuts the anchor 88 at a junction region 89 so that the anchor 88 can be separated from the tether 42 once the anchor 88 is deployed in the heart. FIG. 12B shows an example in which the guide arm 82 is pulled proximally over the junction region 89 and the anchor 88 is separated from the tether 42. For example, the guide arm 82 may maintain the position of the anchor 88 adjacent to (e.g., abutting) or connected to the tether 42 while the junction region 89 is within the guide arm 82, but once the guide arm 82 is pulled proximally over the junction region 89, the anchor 88 may be disconnected from the tether 42. In some examples, the proximal end of the anchor 88 and the distal end of the tether 42 include matching shaped interfacing features 43a and 43b, respectively, to increase engagement of the anchor 88 and the tether 42 within the guide arm 82. In some examples, the tether 42 is releasably attached to the anchor 88 using one of the releasable connectors described in WO 2022/046678. Axial movement of tether 42 and anchor 88 may be controlled by an actuator 90 in the proximal controller. Axial movement of the anchor 88 relative to the guide arm 82 may be useful, for example, in changing the shape of the guide arm 82, as described herein.

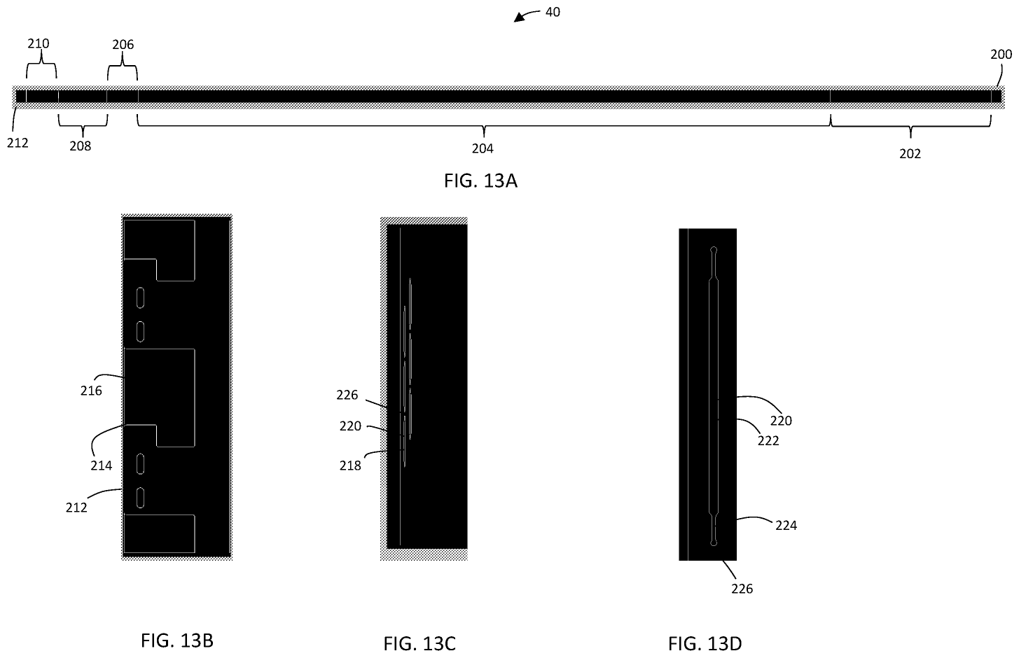

As shown in FIG. 13A, rotation control shaft 40 extends from a proximal end 200, where it connects to actuator 80, through a proximal region 202, an IVC region 204, an OS region 206, an IS region 208, and a distal region 210 to a distal end 212, where it connects to guide arm 82. FIG. 13B shows a pattern of laser cut lines 214 (shown in white against the black background) to remove material from areas 216, thereby creating a connector at the distal end of distal region 210 to engage a mating connector at the proximal end of guide arm 82 to form a joint. The laser cut pattern in the remaining portion of the distal region 210 (not shown in the figures) is in the form of cut segments disposed in a spiral.

IS region 208 corresponds to the portion of rotation control shaft 40 that will be disposed within the distal region of inner steerable catheter 36. FIGS. 13C and 13D show a pattern of laser cut lines 218 (shown in white against the black background) to remove material, thereby creating openings 220 extending circumferentially about IS region 208 of rotation control shaft 40. As shown in the detail of FIG. 13D, each opening 220 has a wide portion 222 and two narrow portions 224. Adjacent rings of openings 220 are offset so that the center of each opening in one ring aligns with the uncut portions 226 in an adjacent ring. While FIG. 3C shows only two partial rings of openings, the openings 220 occupy the entire IS region 208.

OS region 206 has cuts in a pattern identical to that of distal region 210:336 cut segments disposed in a spiral at a first pitch, each cut segment having a first kerf, a first length and a first separation from an adjacent cut. IVC region 204 has a spiral cut pattern that differs from that of OS region 206 and distal region 210. IVC region 204 extends 43 inches and has a pattern of cut segments disposed in a spiral at a second pitch (e.g., larger than the first pitch), each cut segment having the first kerf, a second length (e.g., smaller than the first length) and a second separation from an adjacent cut (e.g., larger than the first separation).

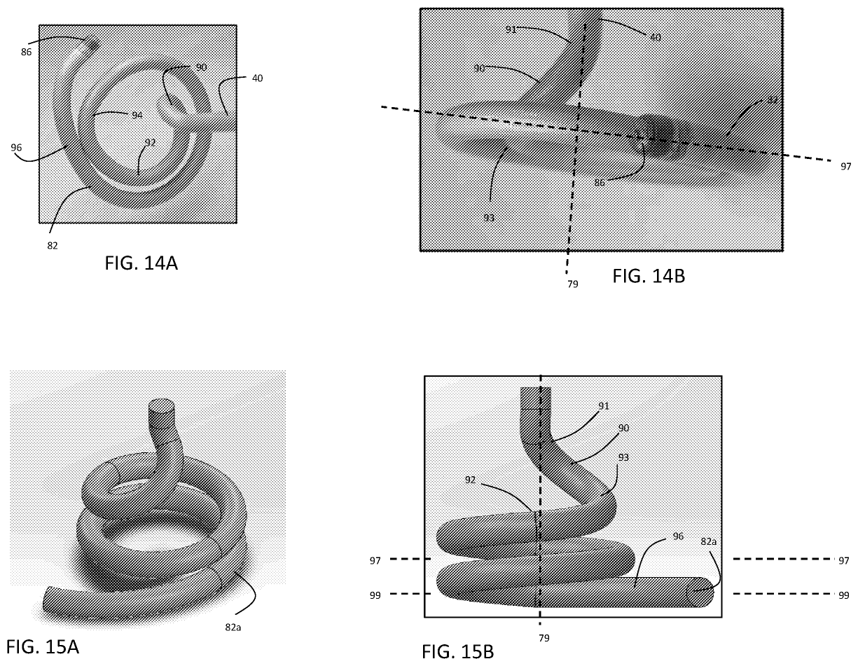

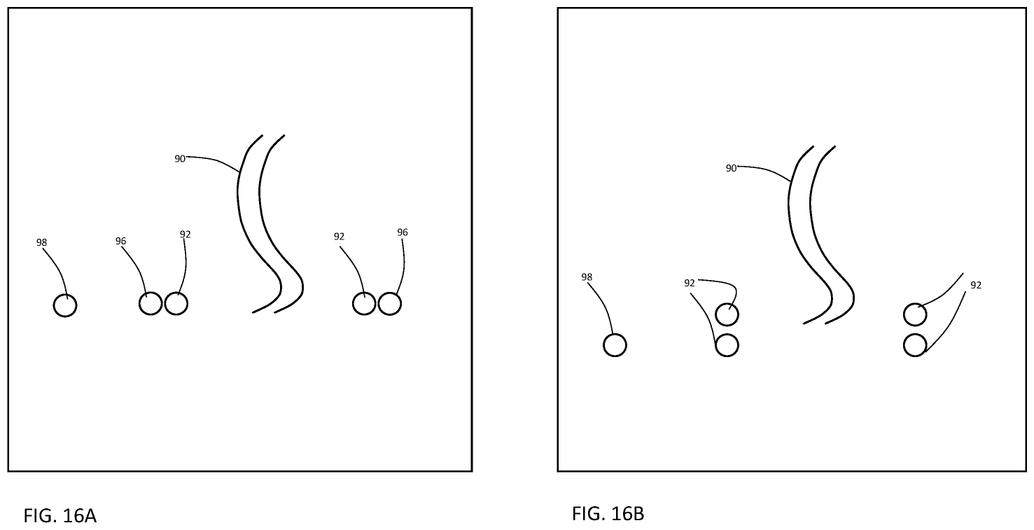

FIGS. 14A-15B are embodiments of guide arm 82 and the distal end of rotation control shaft 40. As shown, guide arm 82 is in a spiral configuration (FIGS. 14A and 14B) and/or helical configuration (FIGS. 15A and 15B), such as the configuration it would controllably assume after emerging from the distal end of the inner steerable catheter in the left atrium of the heart. The geometry of the guide arm 82 provides a consistent self-assembly with a proven encircling geometry. As will be described in more detail, the anchor control catheter is used as the encircling tool which decouples the anchor itself from (e.g., direct) encircling.

Referring to FIGS. 14A and 14B, guide arm 82 has three parts: A curved part 90 extending radially outwards from a joint at the distal end of the rotation control shaft 40, an intermediate part 92 extending from the curved part 90 to a transition point 94, and a distal part 96 extending from the transition point 94 to the distal end of the guide arm that includes cap 86. The rotation control shaft 40 generally extends axially along a longitudinal axis of the anchor delivery subsystem and three nested catheters. The curved part 90 of the guide arm 82 bends at bend 91 and extends radially outwards before bending again sharply inwards at bend 93 as it transitions to the intermediate part. The distal part 96 and the intermediate part 92 generally lie in a plane 97 that is orthogonal to axis 79 of the rotation control shaft 40 (or the longitudinal axis of the anchor delivery subsystem). The curved part and bends 91 and 93 facilitate the transition of the guide arm 82 from aligning with the longitudinal axis of the rotation control shaft to the proximal and distal parts laying in the plane that is orthogonal to the longitudinal axis. This embodiment of the guide arm 82 is described as having a spiral configuration.

FIGS. 15A and 15B, show a view of another embodiment of a guide arm 82a that has a helical configuration in contrast to the spiral configuration of guide arm 82. As with the embodiment above, the guide arm 82a includes a curved part 90 that extends radially outward intermediate part 92 and the distal part 96 do not lie together in a single plane that is orthogonal to the longitudinal axis of the anchor delivery subsystem or nested catheters. Instead, in this embodiment, the intermediate part comprises a helical section that includes turns (e.g., loops) axially spaced apart (e.g., that reside in more than one plane). As can be seen, depending on the number of turns, this results in the proximal portion turning in a helical fashion so as to lie in at least planes 97 and 99 before transitioning to distal part 96 which lies in plane 99. In the illustrated example, the intermediate part can include, for example, less than two turns. FIGS. 15A and 15B show the helical guide arm 82a, including how the distal and intermediate parts in guide arm 82a lie in planes 97 and 99, in contrast to the in-plane arrangement of guide arm 82 (see FIG. 14B). The geometry of the guide arm, for example the distal part 96 lying in a plane 99 that is generally orthogonal to axis 79 of the rotation control shaft, facilitates delivery of the anchor into a planar arrangement with the mitral annulus. The anchor delivery system is designed so as to maintain the orthogonal arrangement of the guide arm to the rotation control shaft throughout the delivery procedure, which, along with independent user control of system rotation, grabber reach, and system (axial) position, enables repeatable and fine-tuned control of depth/radial extent to the clinician during encircling.

Since the guide arm is not implanted or left behind in the patient, inclusion of visualization features or markers thereon that facilitate imaging in real-time such as with ultrasound and/or fluoroscopy can be made without concern for the impact such features would have on implant (e.g., anchor) delivery or performance. For example, the guide arm can include radiopaque markers to allow for this visualization. Additionally or alternatively, the construction of the anchor control catheter with laser cut shape memory or nitinol tubing provides highly reflective features that are easily visualized via ultrasound.

FIG. 16A is a representation of a sample cross-sectional echo image of the guide arm 82 from FIGS. 14A and 14B. As shown in FIG. 16A, the curved part 90 is visible under ultrasound along with circular cross-sections in a single plane representing the intermediate part 92, distal part 96, and distal tip 98 of the guide arm 82. FIG. 16B is a representation of a sample cross-sectional echo image of the guide arm 82a from FIGS. 15A and 15B. In this image, since the intermediate part 92 makes more than one helical turn, the intermediate part rests in more than one plane, so the circular cross-sections of the intermediate part 92 are readily visualized and distinguished in the ultrasound image. Additionally, this allows for easier visualization of the distal tip 98 as it is distinguishable as a single circular cross-section spaced apart from the paired cross-sections of the helical intermediate part. This results in easier visualization of the distal tip of the guide arm 82a, which can help the user to encircle selected anatomy with the distal tip since it is more distinct on echo imaging.

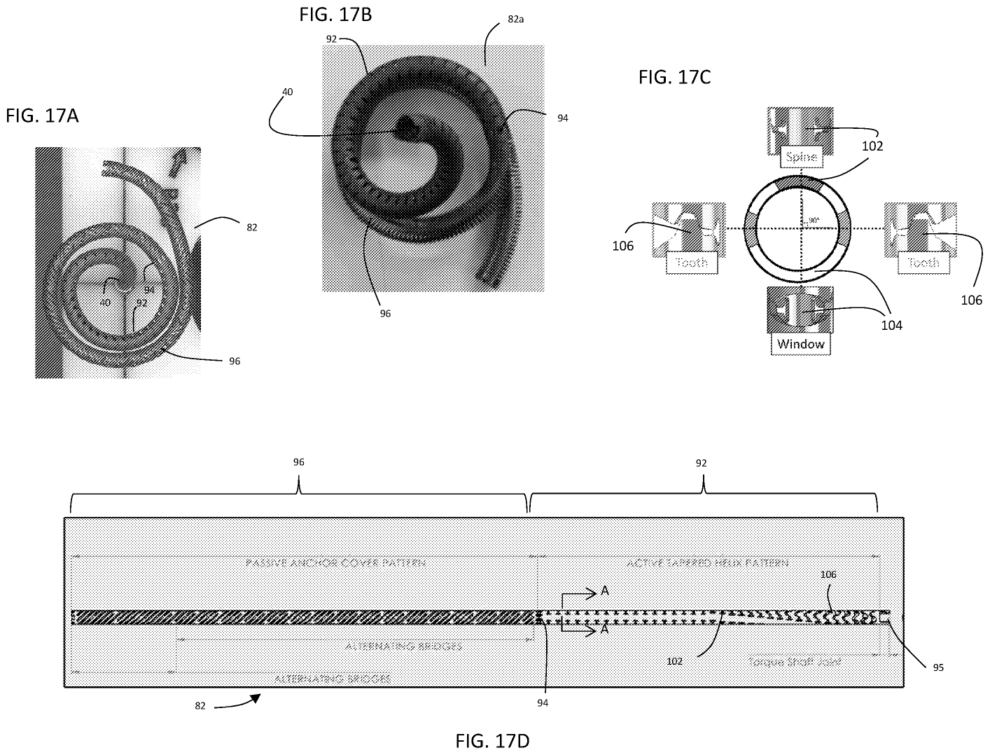

Referring to FIGS. 17A-17D, the guide arm can be a shape memory material laser cut in a combination of active (e.g., 92) and passive (e.g., 96) sections, with transition region 94 and a tether coupling portion 95. Active section 92 can comprise a tapered helix pattern with a longitudinal spine 102 extending generally helically in a proximal portion thereof, and longitudinally in a distal portion of the active section 92. A series of windows 104 can be disposed opposite to spine 102, and a pair of toothed sections 106 are disposed 90° apart from the windows 104 and spine 102. In some embodiments, passive section 96 comprises a generally spiral cut pattern with periodic bridge structures. In some embodiments passive section 96 comprises a longitudinal spine (or spines) with spaces (or cuts) disposed on radially inward and/or outward aspects of the guide arm (providing radial flexibility and axial stability). This combination of these active and passive sections, and the preset (shape memory) shape, of cause the guide arm to assume a spiral (e.g., 82, FIG. 17A) or helical (e.g., 82a, FIG. 17B) shape (depending on if the configuration of FIGS. 14A and 14B is used or the configuration of FIGS. 15A and 15B) when it emerges from the steerable catheter. Proximal movement of actuation catheter 39 with respect to guide arm 82 (FIG. 12) engages opposed edges of the tapered helix cut pattern to lock the active section 92 into the desired shape, i.e., extending from the longitudinal axis of the inner steerable catheter through an approximately 90 degree bend to the flat spiral or helical shape described above. Since the shape of the anchor control catheter is formed by a shape memory or nitinol laser cut tube, the complex surface features reflect acoustically quite well and enable distinctive echo visualization.

FIG. 17C shows example aspects for a helical (e.g., FIG. 17B) guide arm. As shown, a series of windows 104 may be disposed opposite to spine 102, and a pair of toothed sections 106 are disposed 90 degrees apart from the windows 104 and spine 102. This configuration, and the preset shape, of proximal part 92 causes it to assume a helical shape when it emerges from the steerable catheter. Proximal movement of actuation catheter 39 with respect to guide arm 82 engages opposed edges of the tapered helix cut pattern to lock the proximal part 92 into the desired shape, i.e., extending from the longitudinal axis of the inner steerable catheter through a 90 degree bend to the flat spiral of the distal part 96.

The distal part 96 of the guide arm 82 can be configured to be manipulated from its set shape into more open and/or more closed shapes by moving tether 42 to provide proximal and distal movement of anchor 88 within distal part 96 of guide arm 82 (FIG. 12). The support structure of distal part 96 can be laser cut in a pattern of alternating spiral components 98 and bridges 100 that provide flexibility so that proximal and distal movement of the stiffer anchor within intermediate part 92 and distal part 96 can bend or straighten distal part 96. The shape of the distal part 96 can also be controlled by movement of actuation catheter 39.

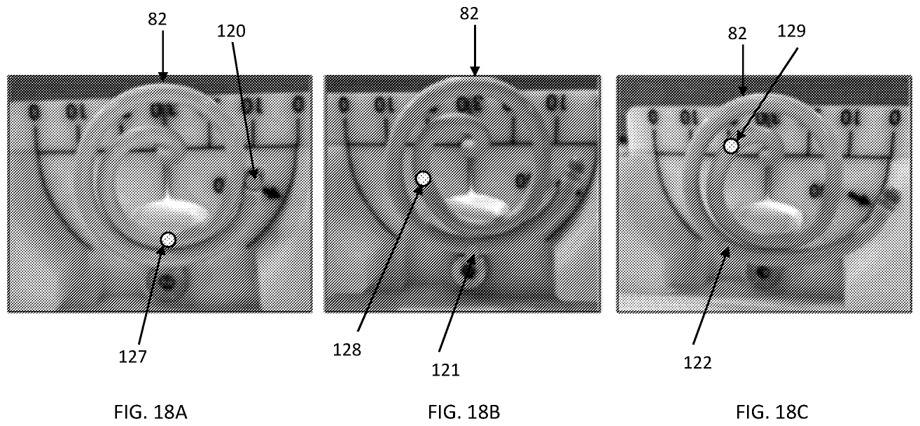

FIG. 18A shows the guide arm 82 in a self-assembly position in which the distal portion of the anchor within the guide arm 82 is deployed to a depth indicated by arrow 120 and the proximal portion of the anchor within the guide arm 82 is deployed to a depth indicated by arrow 127. In the self-assembly position, the shape and depth of the anchor within the guide arm 82 results in the distal end (e.g., tip) and distal part (which may be referred to as a grabber, grabber arm or grabber portion) of the guide arm 82 resting against itself as shown. Such a configuration provides a minimized “envelope” of the guide arm, assisting in: 1) deployment of the guide arm 82 within the atrium, and 2) advancement of the anchor control catheter from the atrium to the ventricle—without deleterious contact/entanglement with native tissue. FIGS. 18B and 18C show the guide arm 82 in an encircling position in which the distal portion of the anchor is retracted within the guide arm 82 to a depth indicated by arrows 121 and 122, and the proximal portion of the anchor is retracted within the guide arm 82 to a depth indicated by arrows 128 and 129, respectively. As shown, retracting the anchor results in the distal end (e.g., tip) and distal part of the guide arm 82 extending radially outwards, while maintaining the proximal portion of the anchor distal to bend 93 of the guide arm 82. That is, movement of the anchor within the guide arm 82 can change a shape (e.g., change a radius of curvature) of the guide arm 82. This allows for fine user control of the angle and distance of the distal end (e.g., tip) from the rest of the guide arm 82 to assist the user with encircling of the chordae. Keeping the proximal portion of the anchor distal to the bend 93 of the guide arm 82 maintains the prescribed geometry of the guide arm 82 while enabling adjustability of the distal end (e.g., tip) of the guide arm 82 during encircling.

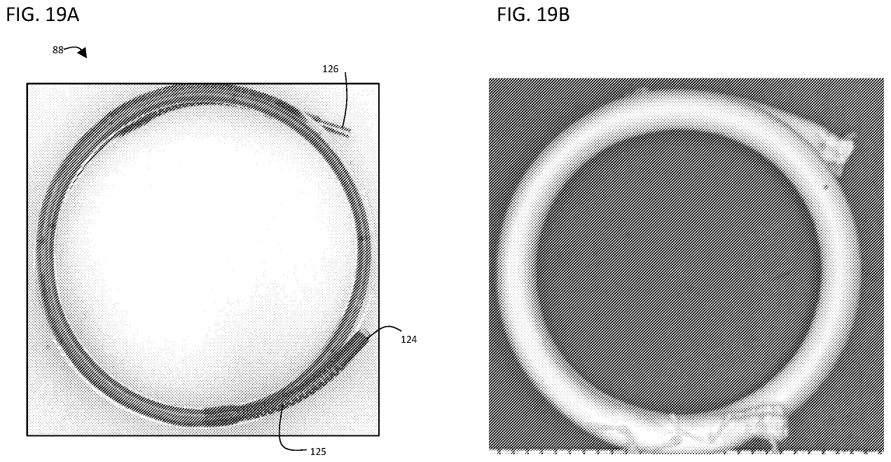

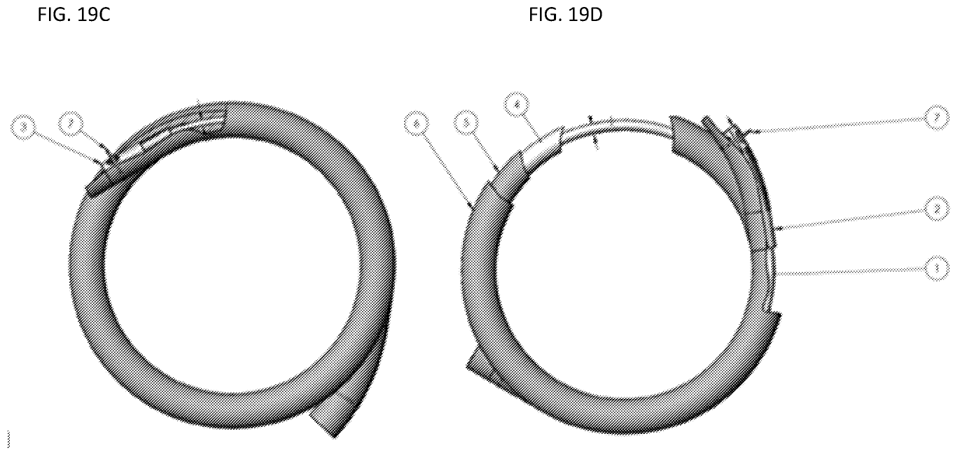

FIGS. 19A-19D show details of anchor 88. When loaded into the anchor control catheter, anchor 88 assumes a generally straightened shape. In its unconstrained state, anchor 88 extends in a spiral from a distal tip 124 to a proximal connector 126, where it releasably attaches to the tether. A laser-cut segment 125 at the distal end is more flexible than a central portion of the anchor and is therefore less traumatic to the delivery system and the patient's anatomy. A flexible laser-cut segment may be provided on the proximal end of the anchor as well. Using the inner steerable catheter 36 for steering under ultrasound and/or fluoroscopic visualization, the spiral portion of the anchor control catheter 38 is then advanced through the leaflets of the native valve.

FIG. 19B shows the anchor of FIG. 19A including one or more layers of expanded polytetrafluoroethylene (ePTFE) disposed over the anchor to provide a lubricious and biologically inert coating that protects the anatomy from damage or abrasions that could otherwise be caused by uncoated metal. While ePTFE is used in this embodiment, it should be understood that other similar materials can be used with the anchor. FIGS. 19C and 19D provide additional cutaway views of the anchor, including a core 1 (e.g., a shapeset material such as nitinol), a distal tip 2, and a proximal termination assembly 3. The anchor can include one or more ePTFE coatings, in this embodiment the anchor includes inner coating 4, middle coating 5, and outer coating 6. Furthermore, the anchor can include suture points 7 on one or both of the proximal and distal tips that can be used to secure the one or more coatings to the anchor.

To deliver an anchor to a patient's heart, the outer steerable catheter 34 and inner steerable catheter 36 are used to navigate the delivery system within a sheath (not shown) to the patient's right atrium RA and through the septum to the left atrium LA. The anchor control catheter includes flexibility and smooth rotational control of the catheter across the septum. The guide arm 82 of the anchor control catheter 38 is then advanced out of the distal end of the inner steerable catheter 36 where it assumes a spiral shape or helical shape under the control of the shape set of the intermediate part of control arm 82 and the control of the distal part of control arm 82 by actuation catheter 39. The anchor also fully self-assembles and assumes its at-rest shape within the guide arm of the anchor control catheter within the left atrium. When positioned with its distal end generally adjacent to the distal end of the anchor control catheter, the smaller radius or profile of the anchor with respect to the distal arm radius drives a smaller self-assembly envelope of the anchor control catheter within the left atrium.

Using the inner steerable catheter 36 for steering under fluoroscopic visualization, the spiral or helical portion of the anchor control catheter 38 is then advanced through the leaflets of the native valve 130 into the left ventricle LV. Using the actuation catheter 39 to extend the distal tip of guide arm 82 radially outwards from the guide arm, the anchor control catheter 38 is rotated within the left ventricle to advance guide arm 82 between the chordae and the heart wall with the anchor still inside the anchor control catheter. Because the anchor is stiffer than the guide arm, as described above, the position of the anchor with respect to the distal part of the guide arm can also be used to conform that portion of the guide arm to the spiral shape of the anchor (e.g., the radius of the anchor). The chordae can then be encircled with the guide arm for at least the full length of the anchor (e.g., approximately 1.5 full turns). Once the anchor and guide arm are in place, one procedural strategy to reduce left ventricular outflow tract obstruction (LVOTO) from the prosthetic valve is to implant the anchor as high as possible within the anatomy. Satisfactory encircling of chordae and/or leaflets can also be assessed via ultrasound and/or fluoroscopy, on account of the echogenic and radiopaque features of the anchor control catheter and/or the anchor. This can be accomplished by lifting up on the anchor prior to pulling the anchor towards the left atrium. In some embodiments, this adjustment is made by pulling on the anchor control catheter, since the anchor still resides within the guide arm. In other embodiments, if the anchor has already been deployed from the guide arm, the anchor can be repositioned or pulled up or towards the atrium with the tether.

To fully deploy the anchor in the left ventricle, the anchor control catheter 38 is withdrawn from the anchor while holding tether 42 stationary. The anchor control catheter 38 is withdrawn into the inner steerable catheter 36 until the distal end clears the proximal end of the anchor. The tether is then decoupled from the anchor, the inner steerable catheter 36 is withdrawn into the outer steerable catheter 34, and the anchor delivery subsystem is withdrawn from the patient. The anchor does not lose chordae and remains stable during anchor control catheter retraction. The anchor control catheter retraction is a simple process.

The anchor delivery subsystem including the anchor control catheter and guide arm described above provides a purpose-built chordal encircling tool configured to deliver the anchor. As described above, the anchor delivery subsystem is configured to safely assemble the anchor within the anchor control catheter in the left atrium and away from the chordal apparatus. The anchor delivery subsystem includes a small envelope with low tip penetration power to avoid contact with the left atrium. The configuration provides a simplified system with only a single tube (e.g., the anchor control catheter guide arm) encompassing the anchor. When encircling within the left ventricle, only the fully self-assembled anchor control catheter touches the chordae, and there is little to no friction stack-up during encircling as can occur in alternative devices.

When the anchor control catheter and anchor are advanced from the left atrium into the left ventricle, the anchor control catheter provides excellent visualization in a single echo plane with standard echocardiographic views in the left ventricle during encircling. As described above, a user can easily visualize the turns of the anchor control catheter with distinct visualization of the intermediate portions and of the tip of the guide arm. Encircling control is enabled in both depth (e.g., inferior/superior) control—by advancing/retracting the anchor control catheter, and independently, reach (e.g., radial) control—by axial movement of the anchor within the anchor control catheter to modify its radial reach at the distal tip thereof. The combination provides a user with fine-tuned adjustability of encircling position within the heart. This visualization combined with control of the tip greatly simplifies the encircling process to navigate a variety of patient anatomies.

Additionally, the encircling process is easily reversable as many times as required to capture the intended chordae and get the anchor in position. If chordae are missed or the user is not satisfied with the position of the anchor control catheter or anchor, both can be repositioned simply by unwinding the encircling and starting the process again. The simplified design of the anchor control catheter and limited cross-sectional diameter further provide stable hemodynamics throughout the anchor delivery process.

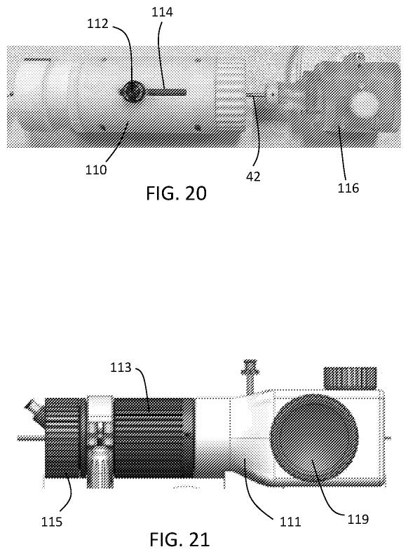

FIG. 20 shows the part of the proximal controller containing the actuators for the anchor control catheter and tether according to an embodiment of the invention. Handle 110 is attached to the rotation control shaft 40 (not shown in FIG. 20) such that rotation of handle 110 rotates shaft 40 and guide arm 82. Slider control 112 extends from actuation catheter 39 through a slot 114. Proximal and distal movement of slider control 112 moves actuation catheter 39 proximally and distally, respectively. A second handle 116 is attached to tether 42. Proximal and distal movement of handle 116 with respect to handle 110 moves tether 42 and the anchor attached to it proximally and distally within the anchor control catheter. Likewise, proximal movement of handle 110 with respect to a stationary handle 116 will cause the anchor to remain stationary as the guide arm 82 retracts, as described below. Depressing a button 117 on handle 116 causes handle 116 to grip tether 42, and releasing button 117 allows tether 42 to move with respect to handle 116.

FIG. 21 is another embodiment of the proximal controller that combines into one handle the actuators for the anchor control catheter and tether. Handle 111 is attached to the rotation control shaft 40 (not shown in FIG. 21) such that rotation of ring 113 rotates shaft 40 and guide arm 82. Proximal and distal movement of ring 115 moves actuation catheter 39 proximally and distally, respectively. Depressing a button 119 causes handle 111 to grip tether 42 so that the tether will move with handle 119.

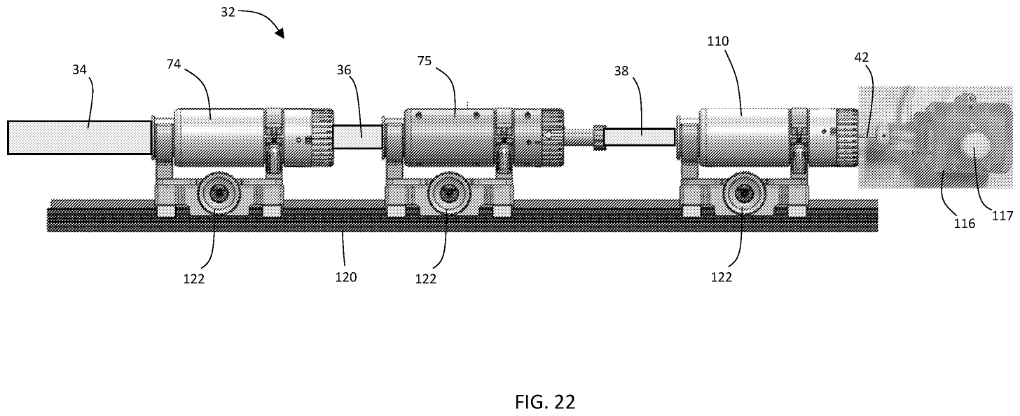

FIG. 22 shows components of the proximal controller 32 for the anchor delivery subsystem. Tether control handle 116 is at the proximal end of controller 32 and is movable with respect to anchor control catheter handle 110 to control relative movement between the anchor control catheter 38 and tether 42, as described above with respect to FIG. 20A. Anchor control catheter handle 110 is mounted on a rail 120 and is movable with respect to the control handle 75 of the inner steerable catheter 36. A lock 122 holds handle 110 in place on rail 120 when the handle is not being moved. Likewise, control handle 75 is proximal to control handle 74 of the outer steerable catheter 34. Locks 122 hold handles 74 and 75 in place on rail 120 when they are not being moved.

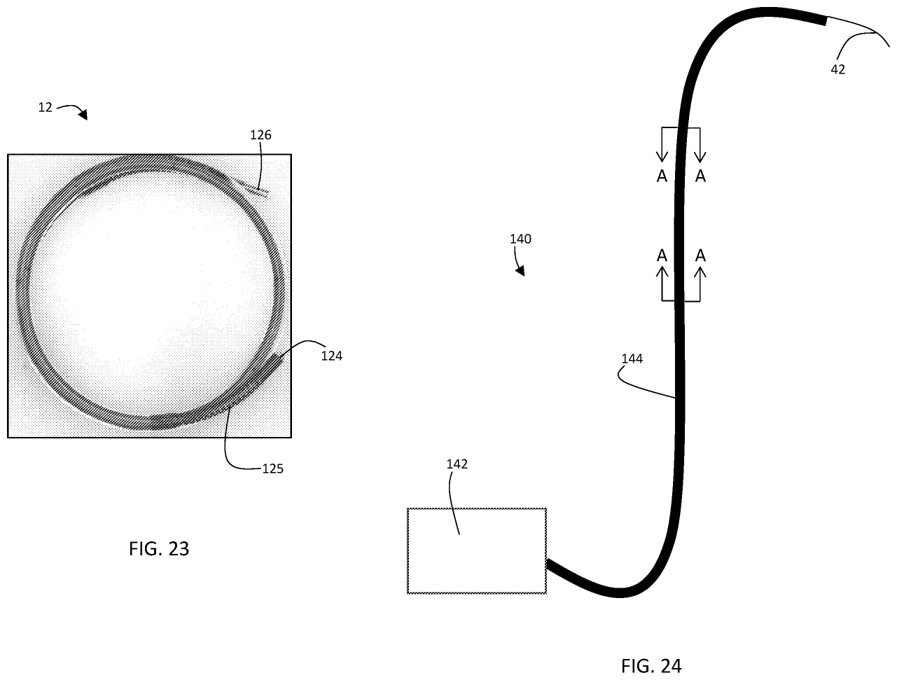

FIG. 23 shows details of anchor 12. When loaded into the anchor control catheter, anchor 12 assumes a straightened shape. In its unconstrained state, shown in FIG. 22, anchor 12 extends in a spiral from a distal tip 124 to a proximal connector 126, where it releasably attaches to the tether. A laser-cut segment 125 at the distal end is more flexible than a central portion of the anchor and is therefore less traumatic to the delivery system and the patient's anatomy. A flexible laser-cut segment may be provided on the proximal end of the anchor as well. Using the inner steerable catheter 36 for steering under fluoroscopic visualization, the spiral portion of the anchor control catheter 38 is then advanced through the leaflets of the native valve 130.

To deliver an anchor to a patient's heart, the outer steerable catheter 34 and inner steerable catheter 36 are used to navigate the delivery system within a sheath 35 to the patient's right atrium RA and through the septum to the left atrium LA. The guide arm 82 of the anchor control catheter 38 is then advanced out of the distal end of the inner steerable catheter 36 where it assumes a spiral shape under the control of the shape set of the proximal part of control arm 82 and the control of the distal part of control arm 82 by actuation catheter 39. Using the inner steerable catheter 36 for steering under fluoroscopic visualization, the spiral portion of the anchor control catheter 38 is then advanced through the leaflets of the native valve 130 into the left ventricle LV. Using the actuation catheter 39 to extend the distal part 96 of guide arm 82 out of the spiral shape, the anchor control catheter 38 is rotated within the left ventricle to advance guide arm 82 between the chordae 132 and the heart wall with the anchor 12 still inside the anchor control catheter. Because anchor 12 is stiffer than guide arm 82, as described above, the position of the anchor 12 with respect to the distal part of the guide arm can also be used to conform that portion of the guide arm to the spiral shape of the anchor (e.g., the radius of the anchor). After the chordae 132 have been encircled for the full length of the anchor, the anchor control catheter 38 is withdrawn from anchor 12 while holding tether 42 stationary. The anchor control catheter 38 is withdrawn into the inner steerable catheter 36, the inner steerable catheter 36 is withdrawn into the outer steerable catheter 34, and the anchor delivery subsystem is withdrawn from the patient while leaving tether 42 connected to anchor 12.

Prior to delivering the expandable valve to the implanted anchor, a guidewire is inserted through the anchor. One technique for placing the guidewire is to advance an inflated balloon catheter through the implanted anchor toward the apex of the heart. The position of the balloon may be monitored with ultrasound. Using a balloon with a large-enough diameter (e.g., >12 mm) helps ensure that the balloon will not pass between groups of chords. Once the balloon has passed through the anchor and into the ventricle, the guidewire can be advanced through the balloon catheter lumen. The balloon catheter is then withdrawn, leaving the guidewire in place for use in advancement of the valve delivery catheters.

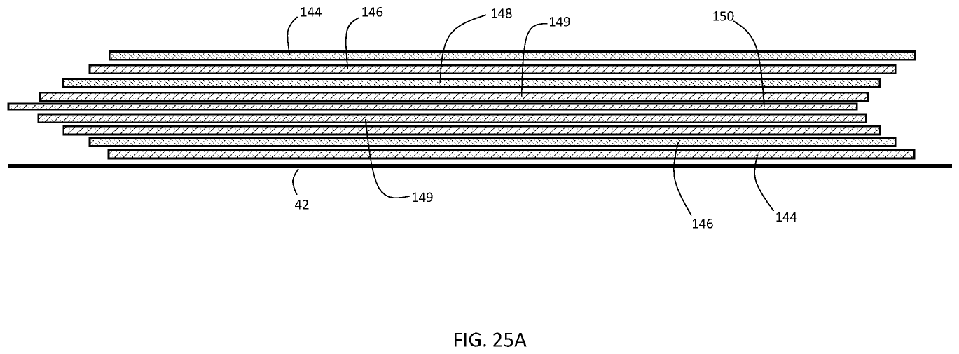

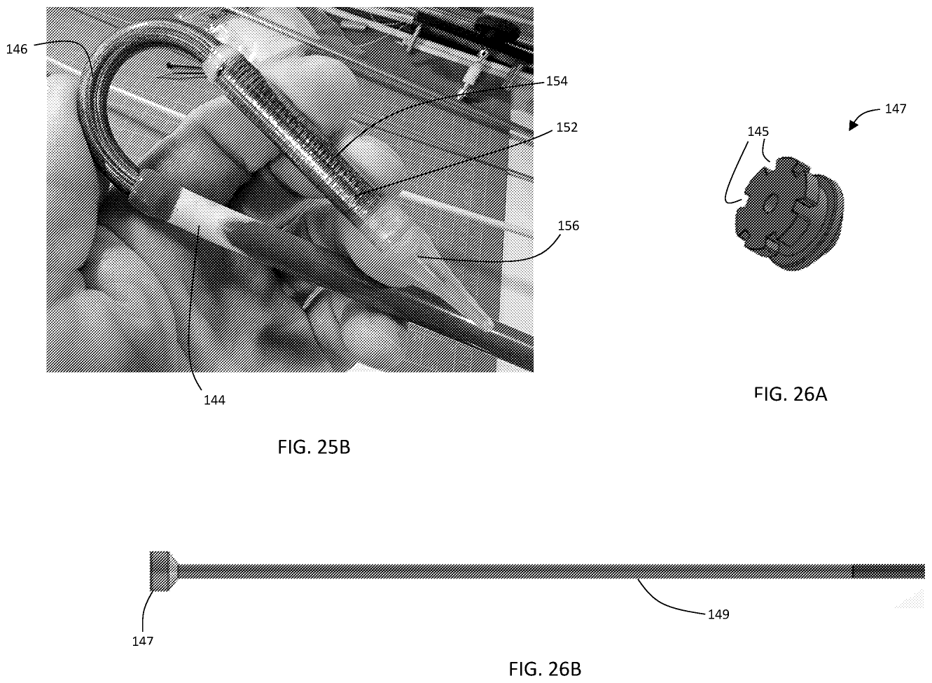

FIGS. 24 and 25A show aspects of a valve delivery subsystem 140 having a proximal controller 142 and three nested catheters (as shown in cross-section in FIG. 25A): An outer steerable catheter 144, a capsule shaft 146 movably disposed in the lumen of the outer steerable catheter 144, and an inner steerable catheter 148 movably disposed in the lumen of the capsule shaft. As shown, the tether 42, which is still extending proximally from the previously-implanted anchor, is disposed outside of outer steerable catheter 144. In some embodiments the tether 42 is disconnected from the anchor and removed from the patient prior to introduction of the valve delivery subsystem 140. Disposed within the lumen of inner steerable catheter 148 is a tab retainer shaft 149, and disposed within the lumen of tab retain shaft 149 is a nose cone shaft 150. A guidewire (not shown) may be disposed in a lumen of nose cone shaft 150. Outer steerable catheter 144 and inner steerable catheter 148 may be configured like the outer steerable catheter and inner steerable catheter described above with respect to the anchor delivery subsystem. In some examples, only one of the outer steerable catheter 144 and the inner steerable catheter 148 is included in the valve delivery subsystem 140, and steering is provided by a single steerable catheter (e.g., the inner steerable catheter 148).

FIG. 25B shows the distal end of the valve delivery subsystem, and FIGS. 26A-26B show details of the tab retainer. In the view of FIG. 25B, capsule shaft 146 is shown extending distally of the distal end of outer steerable catheter 144. A valve capsule 152 containing a compressed prosthetic valve 154 is attached to the distal end of the capsule shaft 146. When loaded in the capsule 152, tabs (not shown) on the proximal (atrial) end of valve 154 are disposed in slots 145 of a tab retainer 147 at the distal end of tab retainer shaft 149, as shown in FIG. 26A. A nose cone 156 extends from the distal end of capsule 152. Nose cone 156 is connected to the nose cone shaft 150 (shown in FIG. 25A but not shown in FIG. 25B).

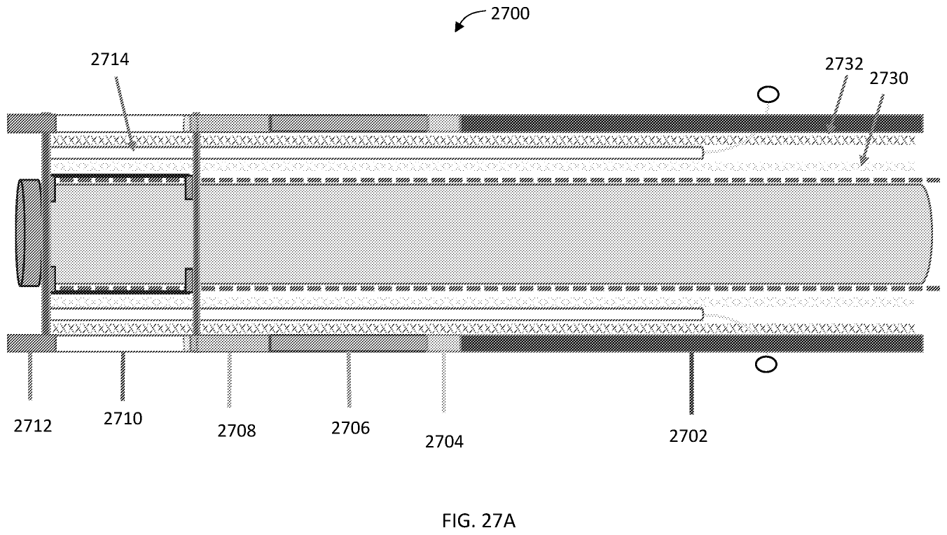

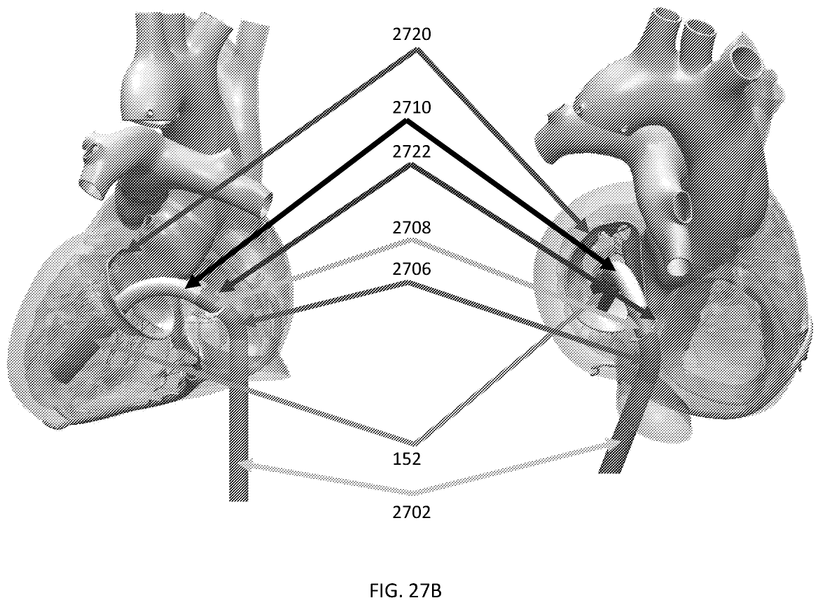

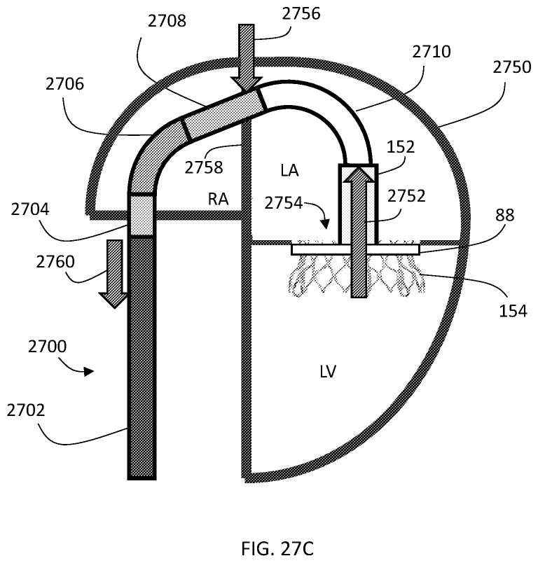

In some examples, the valve delivery subsystem includes a single steerable catheter as opposed to multiple steerable catheters (e.g., an outer steerable catheter 144 and an inner steerable catheter 148 in FIG. 25A). FIGS. 27A-27C show an example of a steerable catheter 2700 that may be used as a single steerable catheter as part of a valve delivery subsystem. As shown in FIG. 27A, the steerable catheter 2700 includes a proximal section 2702, a pivot transition section 2704, a pivot section 2706, a reach section 2708, a steering section 2710 and a tip section 2712. Each of the sections 2702-2712 may comprise one or more materials (e.g., polymer(s)) that provide different degrees of stiffness. For example, the proximal section 2702 may have the greatest stiffness of the sections 2702-2712, the pivot section 2706 may have the least stiffness of the sections 2702 2712, and the steering section 2710 may have a stiffness that is intermediate to those of the proximal section 2702 and the pivot section 2706. The pivot transition section 2704

In some examples, the pivot section 2706 may have a durometer ranging from 35D to 60D (e.g., 35D, 40D, 45D, 50D, 55D or 60D). The pivot transition section 2704 acts as a bridge between the proximal section 2702 and the pivotable section 2706 and has a stiffness that is between that of the proximal section 2702 and the pivotable section 2706 to reduce stress on the transition between the two sections. In some examples, the pivot transition section 2704 may have a durometer ranging from 60D to 80D (e.g., 60D, 65D, 70D, 75D or 80D.). The reach section 2708 acts as a bridge between the proximal section 2702 and the pivotable section 2706 and has a stiffness that is greater than that of the pivot section 2706 and less than that of the pivot transition section 2704 to reduce stress on the transition between the two sections. In some examples, the reach section 2708 may have a durometer ranging from 50D to 70D (e.g., 50D, 55D, 60D, 65D or 70D.). The tip section 2712 may have a stiffness that is greater than that of the pivot section 2706 and less than that of the pivot transition section 2704 and the reach section 2708. The tip section 2712 may be configured to abut or be coupled to a valve capsule, which carries the prosthetic valve therein In some examples, the tip section 2712 may have a durometer ranging from 40D to 65D (e.g., 40D, 45D, 50D, 60D or 65D.).

The steerable catheter 2700 includes two longitudinal pull lumens 2714 placed and braid layers 2730 and 2732, similar to the examples of FIGS. 4A and 4B. For example, pulling one of the pull lumen 2714 causes the catheter 2700 to bend in one direction, and pulling the other pull lumen 2714 causes the catheter 2700 to bend the opposite direction. In this way, the steerable catheter is configured to bend laterally along a plane.

The varied axial stiffness of the steerable catheter 2700 allows the steerable catheter 2700 to take on a desired shape during various parts of the valve delivery. FIG. 27B shows an example of the steerable catheter 2700 in a deflected (e.g., flexed or bent) configuration when steering a valve capsule 152 through the mitral valve annulus 2720 of heart. The catheter 2700 preferably bends at the pivot section 2708 and the steering section 2710 because of the relative flexibility of these sections. As shown, steerable catheter 2700 may be arranged such that the pivot section 2706 is at or near the fossa ovalis 2722 and the steering section 2710 points the valve capsule 152 in alignment with the steering section 2710.

FIG. 27C shows a schematic diagram of the steerable catheter 2700 in a deflected configuration within a heart 2750 showing how the steerable catheter 2700 can provide mechanical advantages and is adapted to reduce forces applied to tissues of the heart 2750. In the example shown, the steerable catheter 2700 is used to pull the lower portion of the prosthetic valve 154 “up” toward the mitral valve annulus 2754 of the heart 2750 to assure good positioning of the anchor 88 and/or prosthetic valve 154. The prosthetic valve 154 is partially expanded from the valve capsule 152 such that the lower portion of the prosthetic valve 154 is expanded within the left ventricle LV. As shown, the bend of the steering section 2710 is within the left atrium LA and the bend of the pivot section 2706 is within the right atrium RA. A valve pull up force 2752 is applied on the lower portion of the prosthetic valve 154 and the anchor 88. This causes a first reaction force 2756 to act on the septum 2758 and a second reaction force 2760 to act on the catheter 2700. The reaction forces 2756 and 2758 acting on septum 2758 and catheter 2700 reduces amplitude of both forces. The reach section 2708 can act as lever and the pivot section 2706 can act as fulcrum such that the septum 2758 gains mechanical advantage. The higher bending stiffness of pivot section 2706 also contributes to reduced forces on septum 2758. For example, the stiffness of the pivot section 2706 can have some give so that a bend angle of the pivot section 2706 can vary slightly as force is applied. This can reduce the amount of pressure placed on the septum 2758, thereby reducing (e.g., preventing) damage to the septum 2758.

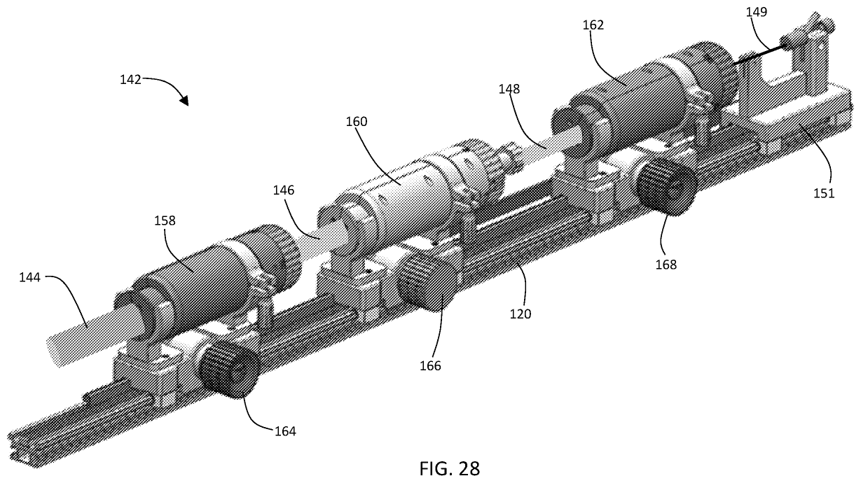

FIG. 28 shows an example of a proximal controller 142 for the valve delivery subsystem having an outer steerable catheter (e.g., FIG. 25A). The tab retainer shaft 149 and nose cone shaft 150 within it are disposed on a carriage 151 mounted on a rail 120 at the proximal end of proximal controller 142. The guidewire described above may be inserted into nose cone shaft 150. Control handles 158, 160, and 162 for the outer steerable catheter 144, capsule shaft 146, and inner steerable catheter 148 are also movably mounted on rail 120. Locks 164, 166, and 168 hold the control handles in place on rail 120 when they are not being moved.

The valve delivery subsystem is placed into the patient's vasculature through the same femoral vein introducer sheath used for the anchor delivery and implantation. To advance the valve capsule to the heart, the control handles 158, 160, and 162, and carriage 151, are advanced together along rail 120 under fluoroscopic guidance. During navigation of the prosthetic valve through the vasculature, the distal end of the valve delivery subsystem is steered by bending the distal ends of inner and outer steering catheters 148 and 144 using control handles 162 and 158, respectively, as described above with respect to the inner and outer steering catheters of the anchor delivery subsystem. The valve capsule 152 and nose cone 156 are just distal to the distal end of the outer steerable catheter 144 during advancement into the patient's heart.

When the nose cone 156, valve capsule 152, and the distal end of the outer steerable catheter 144 have passed through the septum into the left atrium of the heart, the inner steerable catheter 148 and valve capsule 152 are advanced out of the outer steerable catheter by moving control handles 160 and 162 and carriage 151 distally while keeping control handle 158 stationary to move the valve capsule 152 into position within the previously implanted anchor. Once in position, the capsule shaft 146 may be retracted while keeping the tab retainer shaft 149 and valve 154 stationary to retract capsule 152 and expose the distal end of valve 154, thereby allowing it to begin to self-expand within the anchor. The partially self-expanded valve may be pulled proximally against the anchor to move the valve and anchor closer to the ventricular side of the native valve annulus. Thereafter, the capsule shaft is retracted further to expose the proximal end of valve 154 to allow it to fully self-expand. When the capsule 152 has been retracted sufficiently to expose the slots 145 of tab retainer 147, the tabs on the valve move out of the slots 145 to release the valve 154 from the tab retainer 147. The valve delivery subsystem may then be removed from the patient.

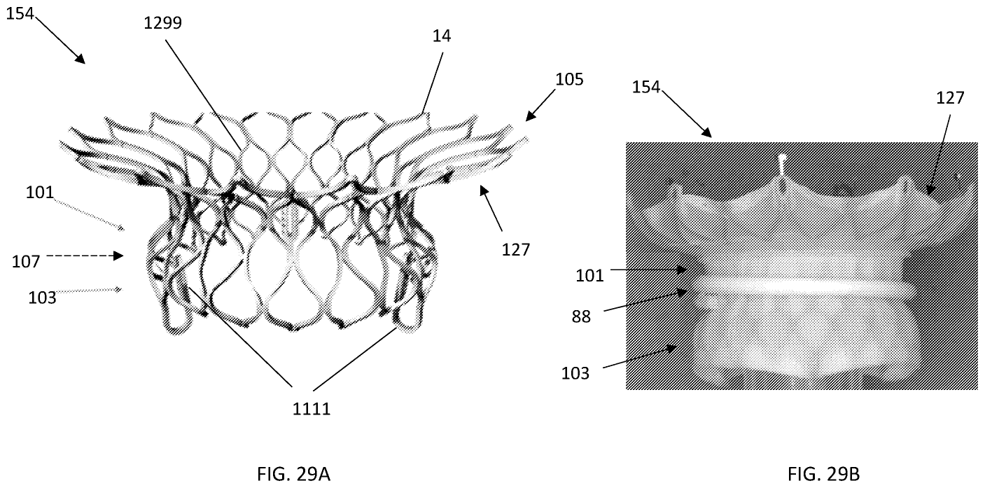

FIGS. 29A and 29B show a valve prosthesis 154 having a valve frame structure 14 and being configured to support a plurality of leaflets (not shown) therein. As described above, the valve prosthesis can be delivered into the deployed anchor with the valve delivery subsystem described above. The valve frame 14 can include interior commissure attachment mechanisms 1111 for attachment of the leaflets to the frame structure 14. The valve frame structure 14 can be deployed from a collapsed (delivery) configuration via the valve delivery subsystem to an expanded configuration during a procedure for replacing or repairing a native valve, such as a mitral valve. The valve frame 14 can include a plurality of rows (e.g., 3-7 rows) of substantially diamond-shaped cells 1299. The valve frame structure 14 can be configured to foreshorten during delivery (i.e., as the valve frame structure 14 transitions from the collapsed configuration to the expanded configuration) due to the cell structure. In some embodiments, the valve frame structure 14 can be configured to self-expand from the collapsed configuration to the expanded configuration (e.g., can be made of a shape memory material such as nitinol). The valve frame 14 can provide circumferential strength and/or longitudinal strength to valve prosthesis 154.

The valve prosthesis 154 can be deployed in an expanded configuration according to the methods described herein. In the expanded configuration, valve prosthesis 154 can be positioned and/or anchored at a target region of a subject (e.g., an organ or tissue of an animal such as a dog, cat, horse, or human). For example, valve prosthesis 154 can be positioned in the expanded configuration in the orifice of a heart valve, such as the mitral valve or tricuspid valve (e.g., to function as a temporary or permanent replacement for an existing mitral valve or tricuspid valve of the heart).

One or more portions of the valve frame structure 14 can be shaped or configured to aid in securing the valve frame structure 14 at a location (e.g., in the orifice of a native heart valve). For example, the valve frame structure 14 can include an atrial flared portion 127 and a ventricular portion 103 configured to help secure the frame in the anatomy. The atrial flared portion 127 and ventricular portions 103 can extend radially outwards from a narrow central waist portion 101. The atrial flared portion 127 can, for example, be configured to extend into the atrium of the heart from the central waist portion 101 when the valve prosthesis is deployed in the native mitral valve. The ventricular portion 103, in turn, can extend into the ventricle of the heart from the central waist portion 101 when the valve prosthesis is deployed in the native mitral valve. The narrow central waist portion 101 is configured to engage with the anchor previously described. The atrial flared portion 127 and ventricular portion 103 can, for example, be configured to be positioned on either side of the anchor 88 (e.g., that is wrapped around the chordae and the central waist portion) to anchor the valve frame structure 14 in the anatomy. Alternatively or additionally, the atrial flared portion 127 and ventricular portions 103 can be configured to engage with tissue to prevent the valve prosthesis from slipping through the native valve orifice.

Referring to FIG. 29A, the frame structure 14 of the valve prosthesis 154 can be in a partial hourglass shape such that the ventricular portion 103 initially flares radially outwards from the central waist portion 101 in the ventricular direction (e.g., above 107), but then curves back inwards towards a center of the frame structure (e.g., below 107). As shown, the interior commissure attachment mechanisms 1111 can be positioned on an interior of the ventricular end of the ventricular portion 103. In some embodiments, the interior surface of the interior commissure attachment mechanisms 1111 can be configured to align radially with the narrow central waist portion 101. This half-hourglass or cup shape of the ventricular portion 103 can advantageously help provide space for the chordae therearound. The ventricular portion 103 is designed to be as short as possible (e.g., having a length of approximately 8 mm to 15 mm in the axial direction) while still achieving the purpose of supporting the attachments to the leaflets and avoiding the commissures/chordae. Additionally, the axial length (or shortness) of the ventricular portion specifically designed to avoid or prevent left ventricular outflow tract obstruction (LVOTO).

As shown in FIGS. 29A-29B, the flared atrial portion 127 also flares radially outwards from the central waist portion in the atrial direction terminating at the wide atrial brim 105. As shown, the atrial brim 105 of the flared atrial portion 127 may curve slightly inwards from the rest of the atrial flared portion, but still points radially outwards from the frame structure 12. The flared atrial portion 127 including the atrial brim is the widest portion of the frame structure, extending out radially further than the ventricular portion. In general, as shown in FIGS. 29A-29B, the atrial flared portion 127 can extend further radially outwards than the ventricular portion 103. Having a larger atrial flared portion can help prevent para-valvular leakage (PVL). The atrial brim 105 can be extremely conformable and compliant to rest against the anatomy without damaging the tissue while also providing sealing without requiring a PVL guard or other additional structure for sealing. Additionally, the size and conformability of the flared atrial portion and atrial brim allows the valve frame structure to be used across large range of anatomies and conditions.

The specific design and shape of the frame structure 12, including the flared atrial portion 127 and the central waist portion 101, and the interaction between the frame structure 14 and the anchor 88, acts to properly seat the frame structure in the atrium. Specifically, when the anchor is placed in the target anatomy (e.g., in the left ventricle, encircling chordae, and positioned “high” near the annulus), engagement between the central waist portion 101 and the anchor 88 acts to pull the frame structure 12, and particularly the flared atrial portion 127 and wide atrial brim 105 “down” toward the native valve to seat the prosthetic valve and form a seal.

The stiffness and compliance of the atrial brim is optimized to assist in expansion of the overall frame structure into the anchor. The flared atrial portion and particularly the atrial brim needs to be sufficiently stiff to tolerate (e.g., initially) non-ideal anchor placement and still achieve full valve expansion. Non-ideal anchor placement can include the anchor being positioned i) at an axial position along the frame other than at the waist, and/or ii) at an angle with respect to the valve frame. The strut or cell patterns of the flared atrial portion have been designed and configured to increase stiffness of the atrial brim to overcome these positioning cases while still allowing the atrial brim to be compliant enough to conform to the anatomy in an atraumatic manner.

In some embodiments, the atrial flared portion 127 is the softest, most compliant, most conformable, or least stiff portion of the valve prosthesis while still having the stiffness required to assume the fully self-expanded configuration when placed within the anchor. This flexibility allows the atrial portion to conform to the atrium of the patient. The central waist portion and the ventricular portion can optionally be stiffer than the atrial portion. The stiffness of the central annular portion can aid in its self-expansion to the target diameter and engagement with the anchor. In some embodiments, the central waist portion needs to be able to expand against the counterforce of the anchor. In certain embodiments, the stiffness of the anchor is selected such that, upon valve expansion, the anchor is partly expanded by the valve. For example, the anchor expansion can increase a circumference of the anchor such that a number of turns in the as-delivered state of the anchor is reduced by from about 5% to about 25%. For example, an anchor initially having 1.5 turns, and having approximately 25% of reduction in turns upon valve expansion therein, will following implantation retain about 1.13 turns. It will be appreciated that the number of turns can be reduced by any percent within the aforementioned reduction range. In another aspect, the number of turns of the anchor can be reduced from about 1.75 turns to about 1.5 turns or about 1.2 turns, or from about 1.5 turns to about 1.3 turns or 1.1 turns.

The replacement mitral valve of the present disclosure is purpose built for the mitral position. As described above, the atrial brim is wide and compliant so as to seal against PVL in a variety of patient anatomies without injuring the atrial tissue. The replacement valve prosthesis additionally has a short (e.g., less than 10 mm) ventricular brim to avoid LVOT obstruction in a variety of patient anatomies. Implantation of the valve as described above does not block the native valve, so the system provides good hemodynamics without the need for pacing. The valve frame structure of the present disclosure does not block the native valve until the atrial brim is deployed, at which point the valve is competent.

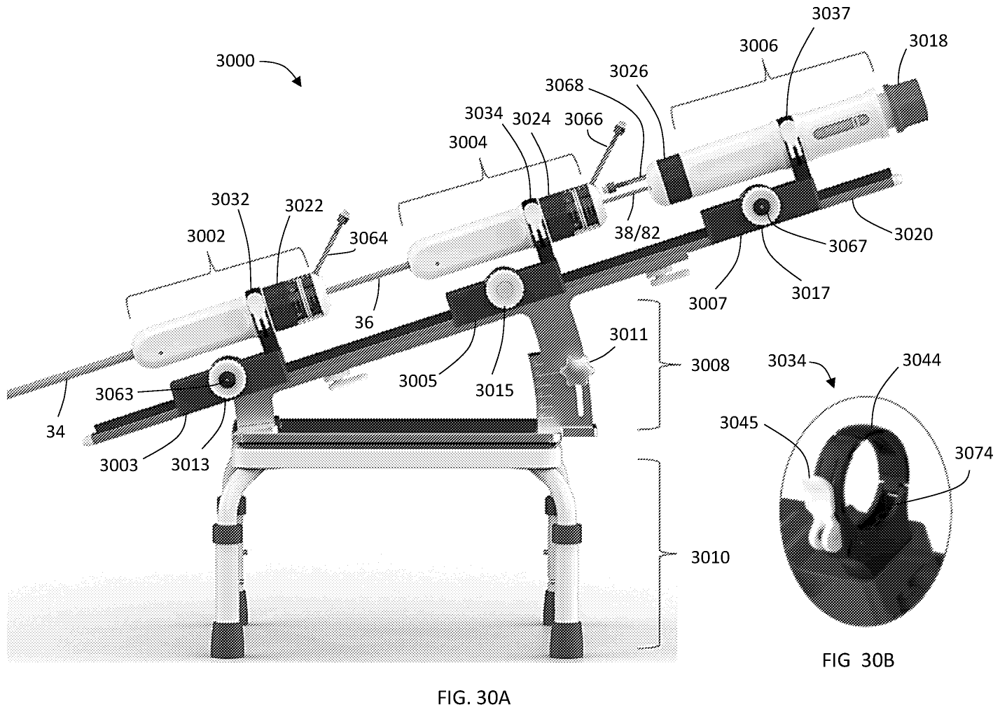

FIGS. 30A and 30B show an example of a proximal controller 3000 for an anchor delivery subsystem. Control handles 3002, 3004 and 3006 are moveably mounted on a rail system 3020 (also referred to as a track system) via carriages 3003, 3005 and 3007, respectively. The rail system 3020 is fixedly coupled to a stabilizer 3008, which may be configured to support the rail system 3020 at an angle with respect to a horizonal axis (e.g., of the floor). In some cases, the stabilizer 3008 may include a knob 3011 (or other angle adjustment device) that is configured to adjust the angle of the rail system 3020 relative to the horizontal axis (e.g., floor). Such adjustment may be based, for example, on the position of the patient, the position of the user, or both. The stabilizer 3008 may include a flat bottom surface for placement on a flat surface of a support 3010, which may be as a stool or table. The vertical height of the proximal controller 3000 may be adjusted by placing the controller 3000 on a support 3010 having a different height, or placing the controller 3000 on an adjustable height support.

The first carriage 3003 includes a first dial 3013 that is configured to be rotated (e.g., by a user's hand) to control distal and proximal movement of a first handle 3002, thereby controlling distal advancement and proximal retraction of an outer steerable catheter 34. The second carriage 3005 includes a second dial 3015 that is configured to be rotated (e.g., by a user's hand) to control distal and proximal movement of a second handle 3004, thereby controlling distal advancement and proximal retraction of an inner steerable catheter 36. The third carriage 3007 includes a third dial 3017 that is configured to be rotated (e.g., by a user's hand) to control distal and proximal movement of a third handle 3006, thereby controlling distal advancement and proximal retraction of an anchor control catheter 38 (the end of which includes the guide arm 82). Thus, the third dial 3017 may be configured to control an axial height of the guide arm 82 within the patient's heart.

In some examples, one or more of the dials 3013, 3015 and 3017 and/or carriages 3003, 3005 and 3007 includes one or more locks to lock translational movement of the handles 3002, 3004 and/or 3006. This may act as a safety feature to prevent unintentional advancement and/or retraction of the outer steerable catheter 34, the inner steerable catheter 36 and/or the anchor control catheter 38, for example when in the patient's body. In some cases, the default state of one or more of the dials 3013, 3015 and 3017 is to be locked such that it/they must be activated to be unlocked. For example, the dial 3013, 3015 and/or 3017 may be configured to be unlocked by pressing the of the dial 3013, 3015 and/or 3017 (or a portion of the dial 3013, 3015 and/or 3017) inward toward the rail system 3020 before the user is able to rotate the dial 3013, 3015 and/or 3017.

One or more of the dials 3013, 3015 and 3017 may be configured to provide independent or coordinated motion with one or more of the other dials 3013, 3015 and/or 3017. For example, when in an independent mode, the second dial 3013 may be configured to allow independent translation of the second carriage 3005 with respect to the first carriage 3003 and/or the third carriage 3007; and when in a coupled mode, the second dial 3013 may be configured to couple translational movement of the second carriage 3005 with translation of the first carriage 3003 and/or the third carriage 3007. In this way, the catheters 34, 36 and/or 38 may be selected to be advanced and/or retracted independently or together. This may be useful in procedures that require independent translation of catheters 34, 36 and 38 during one or more parts of the anchor deployment process, but require coordinated movement between two or more of the catheters 34, 36 and 38 during one or more other parts of the anchor deployment process.

For example, the first dial 3013 of the first carriage 3003 includes a button (e.g., first button) 3063 that is configured to couple translational movement of the first carriage 3003 with translational movement of the second carriage 3005. When the button 3063 is activated (e.g., by pushing), rotation of the second dial 3015 of the second carriage 3005 causes both the first carriage 3003 and the second carriage 3005 to translate along the rail system 3020. Likewise, the third dial 3017 of the third carriage 3007 includes a button (e.g., second button) 3067 that is configured to couple translational movement of the third carriage 3007 with translational movement of the second carriage 3005. When the button 3067 is activated (e.g., by pushing), rotation of the second dial 3015 of the second carriage 3005 causes both the second carriage 3005 and the third carriage 3007 to translate along the rail system 3020. When both the buttons 3063 and 3067 are activated, rotation of the second dial 3015 of the second carriage 3005 causes all three of the first carriage 3003, the second carriage 3005 and the third carriage 3007 to translate along the rail system 3020.

One example in which coupled axial movement of the catheters 34, 36 and 38/82 may be useful is when the catheters 34, 36 and 38/82 are advanced together through the septum and into the atrium of the patient's heart. When such coupled movement is desired, the buttons 3063 and 3067 may be activated and the dial 3015 may be rotated to advance the catheters 34, 36 and 38/82 together in the heart. The dial 3015 may also be rotated (in the opposite direction as advancing) to retract the catheters 34, 36 and 38/82 together out of the atrium of the heart. Fasteners 3032, 3034 and 3037 are configured to secure the handles 3002, 3004 and 3006, respectively, to the rail system 3020. One or more of the fasteners 3032, 3034 and 3037 may also be configured to allow for rotational adjustment of the corresponding handles 3002, 3004 and 3006. For example, as shown in the closeup view of fastener 3034 in FIG. 30B, the fastener 3034 includes a band 3044 (or ring) that is configured to surround an outer surface of the handle 3004. When the lever 3045 is moved to a locked position (e.g., by pressing the lever 3045 and causing the lever 3045 to pivot inward toward the band 3044), tension is applied on the band 3044, thereby constraining movement of the handle 3004 positioned within the band 3044. When the lever 3045 is moved to an unlocked position (e.g., by pulling the lever 2035 and causing the lever 3045 to pivot outward away from the band 3044), the tension is released from the band 3044, thereby allowing rotational movement of the handle 3004. Thus, when the fastener 3034 is in the unlocked position, a user may rotate the handle 3004 by hand to rotate the corresponding catheter 36. In the example of proximal controller 3000, each of the fastener 3032, 3034 and 3037 is configured to allow a user to rotate corresponding handles 3002, 3004 and 3006, thereby allowing rotation of corresponding catheters 34, 36 and 38 (e.g., when in the patient's body).

Each of the fasteners 3032, 3034 and 3037 may be configured to be in an open state in which the respective band is open such that the respective handle may easily be removed from the respective carriage. In addition, each of the fasteners 3032, 3034 and 3037 may be configured transition between a first closed state and a second closed state. For example, when the second fastener 3034 is in the first closed state, the proximal portion (e.g., handle 3004) of the catheter 36 is frictionally secured to the second carriage 3005 so the catheter 36 is maintained at an intended rotational position but is rotatable with respect to the second carriage 3005. For example, the cradle 3074 of the fastener 3034 can include one or more engagement features (e.g., indent (s0, protrusion(s) and/or textured surface(s)) that is configured to frictionally engage with corresponding one or more features of the handle 3004 to maintain the rotational position of the handle 3004 when positioned in the fastener 3034. In the first closed state, the band 3004 may surround the handle 3004 but be loose enough so that the handle 3004 is rotatable with respect to the carriage 3005 (e.g., by a user's hand). When the second fastener 3034 is in the second closed state, the band 3004 is fully sinched down such that the handle 3004 is fully secured to the carriage 3005 and is not rotatable with respect to the carriage 3005.

The handles 3002, 3004 and 3006 include rotational knobs 3022, 3024 and 3026, respectively, that are configured to deflect the distal portions of corresponding catheters 34, 36 and 38. The knobs 3022, 3024 and 3026 can be rotated (e.g., by a user's hand) to deflect the distal portions of the catheters 34, 36 and/or 38 (e.g., each along a single plane), respectively, to steer the catheters 34, 36 and/or 38 through the patient's vasculature. For example, the knob 3024 may be rotated to deflect (e.g., flex) the inner steering catheter 36 to control the position of the anchor control catheter 38/guide arm 82 with respect to the patient's anatomy. For instance, the knob 3024 can be rotated to flex the guide arm 82 toward the patient (“positive flex”) and/or away from the patient (“negative flex”).

The knob 3026 of the third handle 3006 may be rotated to “activate” the guide arm 82 to bias the guide arm to take on the helical or spiral shape (e.g., from a straight shape). Once the guide arm 82 is activated, the knob 3026 may be locked to continuously apply force and maintain the bias on the guide arm 82. In some examples, the guide arm 82 is activated while positioned within the inner steerable, which pre-loads the guide arm 82 such that the guide arm 82 self-assembles when the inner steerable catheter 36 is pulled proximally off the guide arm 82 to expose the guide arm 82. This may be referred to as “active” self-assembly since the guide arm is activated in order to allow the guide arm to self-assemble. Such pre-loading may allow the guide arm 82 to take on the helical or spiral shape within the confines of the atrium with minimal (or no) contact with the inner walls of the atrium.

A proximal knob 3018 of the third handle 3006 may be coupled to the tether (e.g. tether 42), which is coupled to the anchor (e.g., anchor 12) and used to position the anchor relative to the guide arm 82 (distal end of the anchor control catheter 38). For example, the knob 3018 may be rotatable in a first direction to retract the tether/anchor proximally and in a second direction to advance the tether/anchor distally. As described above with respect to FIGS. 18A-18C, the shape of the guide arm 82 (at the distal end of the anchor control catheter 38) may be determined, in part, by the extent to which the anchor is within the guide arm 82. As described previously, controlling distal and proximal movement of the anchor within the guide arm 82 may be used to control the shape of the guide arm 82 during positioning and encircling of the guide arm 82 around the chordae and/or leaflets.

The proximal knob 3018 of the third handle 3006 may also be used to maintain the position of the anchor during retraction of the guide arm 82 (via the anchor control catheter 38) over the anchor within the patient's heart. For example, the proximal knob 3018 may be held fixed (e.g., by the user's hand) and/or locked (using a lock of the knob 3018) to prevent axial movement of the anchor. This may be useful, for example, to hold the anchor steady while the dial 3017 is rotated to retract the guide arm 82 over the anchor. This procedure may be used to ensure that the anchor remains in a desired location and/or orientation around the chordae and/or leaflets as the guide arm 82 is being retracted. For example, this may compensate for any friction between guide arm 82 and the anchor. This may also compensate for any flexibility/compressibility differences between the guide arm 82 versus the anchor.

Each of the handles 3002, 3004 and 3006 may include flush ports 3064, 3066 and 3068, respectively. The flush ports 3064, 3066 and 3068 may provide access to the lumens of respective catheters 34, 36 and 38, for example, for saline flushing.

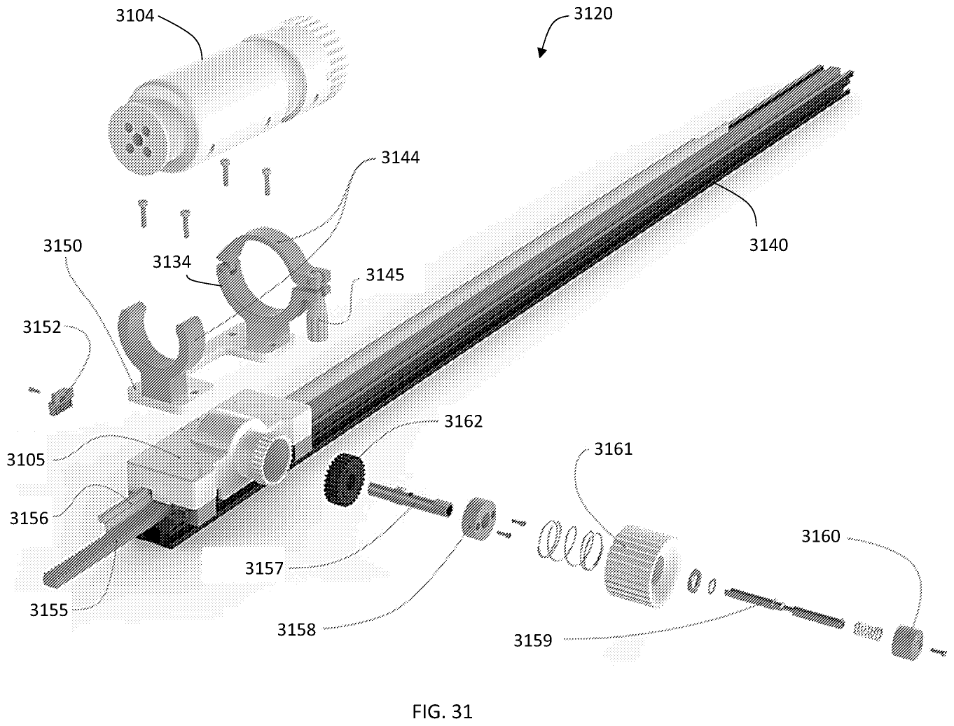

FIG. 31 shows an exploded view of an example rail system 3120 (also referred to as a track system) for a proximal controller to illustrate example features for providing independent and coupled translation of catheters (e.g., catheters 34, 36 and 38). The rail system 3120 includes a primary track 3155 and a secondary track 3156 that are arranged in parallel on a rail 3140. A cradle 3144 is configured to secure a handle 3104 to the track 3140 via a carriage 3105. The cradle 3144 includes a fastener 3134 that includes lever 3145, which is configured to apply and release pressure on a band or ring portion of the fastener 3134. Activation of the lever 3145 constrains movement of the handle 3104, and release of the lever 3145 allows the handle 3104 to be rotated (e.g., by the user's hand). The cradle 3144 may be secured to the carriage 3105 by one or more screws, as shown.

The carriage 3105 includes a knob assembly that is configured to engage with the primary track 3155. The knob assembly includes a gear 3162, an outer shaft 3157, an insert 3158, a rotational knob 3161, an inner shaft 3159 and a button 3160. When the knob 3161 is rotated, the teeth of the gear 3162 engage with teeth of the primary track 3155 to translate the carriage 3105 along to the primary track 3155.

In this example, the carriage 3105 includes a button 3160 that is configured to couple translational movement of the carriage 3105 with another carriage. For example, returning to FIG. 30A, the carriage 3105 may correspond to the first carriage 3003 or the third carriage 3007 that is configured to couple translational movement with the second carriage 3005. Activating the button 3160 (e.g., by pushing the button once) causes the coupler 3152 to engage with the secondary track 3156, thereby coupling translational movement of the carriage 3105 with another carriage (e.g., second carriage 3005). Deactivating the button 3160 (e.g., by pushing the button twice) causes the coupler 3152 to disengage with the secondary track 3156, thereby allowing independent translational movement of the carriage 3105 with respect to the other carriage (e.g., second carriage 3005).

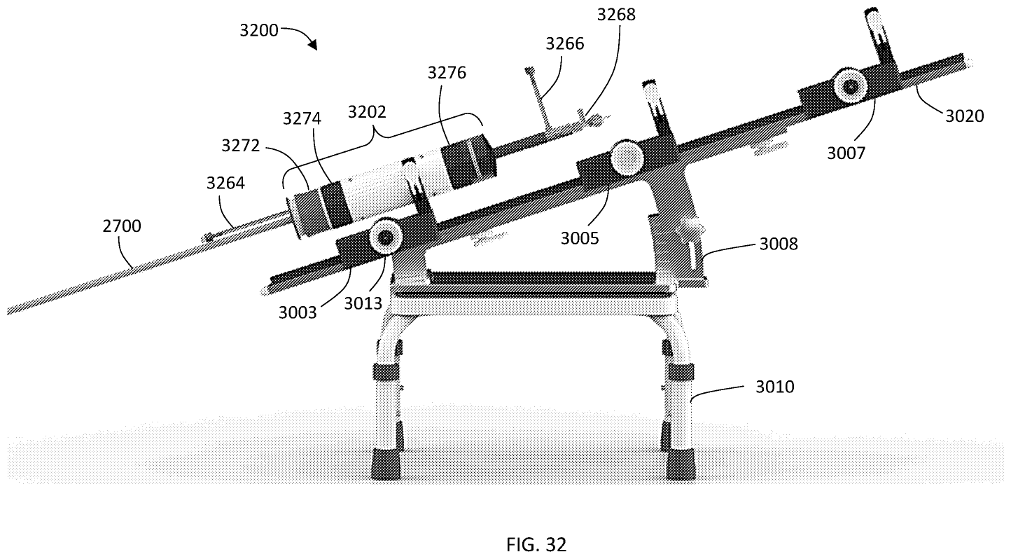

FIG. 32 shows another example of a proximal controller 3200 for a valve delivery subsystem. In this example, the same stabilizer 3008, support 3010, rail system 3020 and carriage 3003 is used as with the proximal controller 3000 of the anchor delivery subsystem in FIGS. 30A-30B. That is, after the anchor is implanted in the patient's heart and the catheters 34, 36 and 38 of the anchor delivery subsystem are retracted out of the patient, the handles 3002, 3004 and 3006 of the anchor delivery subsystem may be removed from respective carriages 3003, 3005 and 3007. A valve delivery handle 3202 may then be positioned in the fasteners 3032 of the carriage 3003 (or carriage 3004 or 3006) for delivery of the valve delivery catheter system, which includes the steerable catheter 2700 with a distal portion having the capsule shaft (e.g., as shown in FIGS. 27A-27C).

The proximal controller 3200 includes a handle 3202, which includes a valve deployment knob 3272, a depth control knob 3274 and a steering/flex shape control knob 3276. The valve deployment knob 3272 is rotatable to cause distal advancement of the prosthetic valve (e.g., 154 in FIGS. 26, 29A and 29B). The valve deployment knob 3272 may include a lock that, when in a locked configuration, prevents deployment of the valve (e.g., as a safety feature). The depth control knob 3274 may be rotatable to cause fine axial movement (e.g., distal advancement or proximal retraction) of the catheter 2700. For example, the depth control knob 3274 may be used during the pulling of the ventricular side of the partially deployed valve toward the native valve annulus, as described herein. The deflection knob 3276 (also referred to as a steering/flex shape control knob) may be rotatable to cause a distal portion of the catheter 2700 to deflect (e.g., flex and change shape).

The dial 3013 of the carriage 3003 may be used to control gross axial movement of the catheter 2700. For example, the dial 3013 may be rotated to introduce the catheter 2700 into the heart and/or advance the catheter 2700 through the septum and into the atrium. The dial 3013 may also be rotated in the opposite direction to retract the catheter 2700 from the heart after the prosthetic valve has been fully deployed. In some cases, the dial 3013 may be used to pull the ventricular side of the partially deployed valve toward the native valve annulus (e.g., instead of or in combination with rotation of the depth control knob 3274).

The handle 3202 may include flush ports 3264, 3266 and 3268. As with the anchor delivery catheter system, the flush ports may be used at various points of the delivery procedure. For example, each of the catheters may be initially flushed to be filled with saline. At various points of the procedure, heparinized saline may be added to combat any clotting in the spaces within and/or between the catheters, frame, anchor, etc.

FIGS. 33A to 33V and 33-1 to 33-13 illustrate example systems and methods for delivering and implanting a prosthetic mitral valve and anchor within a heart of a subject. It should be understood that any of the valves, anchors, anchor delivery subsystems, and valve delivery subsystems described herein may be used in any of a number of combinations, and are not limited by the examples shown in FIGS. 33A to 33V and 33-1 to 33-13. As will be described below, the systems and methods provide a consistent, forgiving delivery procedure that can accommodate a range of patient anatomies, while fully addressing patient valve regurgitation without obstruction of LVOT. As will be apparent from the present disclosure the prosthetic mitral valve and systems and methods of delivery provide a number of clinical benefits and features over other systems on the market.

The valve delivery system of the present disclosure provides forgiving delivery of the anchor transeptally through the native valve and into the left ventricle. The clinician is given fine control of the position of the anchor, and therefore the shape of the guide arm, during encircling, allowing for adjustment of the guide arm radial position to ensure desired chords are captured. While the delivery system provides the ability to capture all of the chords in a single pass (e.g., from between 1 and up to 2.5 rotations of the guide arm), the delivery system also provides the ability to capture only some of the chords in a first revolution (e.g., 1 rotation) of the guide arm/anchor, and to capture the remaining chords on the subsequent revolutions of the guide arm/anchor (e.g., the remaining 1-1.5 rotations). The anchor delivery system provides rotation-based encircling with the ability to reverse and re-encircle the anchor if the clinician is unhappy with device placement or does not capture the desired anatomy within the anchor (e.g., the chords). Since the anchor is (e.g., wholly) contained within the guide arm of the delivery device during encircling, the clinician can easily reverse and re-encircle to safely fix the issue and continue the procedure without having to recapture a deployed anchor. The system is designed and configured to protect the anatomy from chordal injury/rupture.

In addition to being able to control the reach of the guide arm, the clinician is also given full independent control over the axial height of the anchor during and after encircling, as well as the rotational position of the anchor and guide arm. The delivery system and methods disclosed herein further facilitate determination of chordal capture. The position and orientation of the guide arm, and therefore the anchor (carried within), can be visualized with echo (ultrasound) alone during encircling and delivery. This provides for visualization of leaflets traveling outside the guide arm and/or direction visualization of chords. Biplane views can be fixed during the procedure, so the clinician can check leaflet mobility throughout delivery. Visualization of the guide arm also allows for proper alignment of the anchor. The clinician can use the echo visualization to align the (e.g., distal portion of the) guide arm to be co-planar with the annulus. If the clinician achieves balanced capture of the chordae, the guide arm will remain coplanar with the annulus after encircling. An unbalanced or canted guide arm after encircling can indicate to the clinician that additional encircling or re-encircling is required.

In some embodiments, when the anchor is (e.g., fully) deployed from the delivery system into the heart, the anchor is completely released with no tether or other connection to other devices prior to valve deployment. The anchor is stably positioned by circumscribing and gently gathering chordae/leaflets in the left ventricle, while being completely free from (e.g., anchor) delivery system interaction once deployed. The inner diameter of the untethered anchor provides a target through which a guidewire is placed, with the valve delivery system advanced along the guidewire. All of the above features provide fine-tuned control of encircling device, and easy reversibility and safety and re-encircling without undue risk to patient tissue.

The prosthetic valve of the present disclosure also provides a number of advantages over competitors and clinical benefits to the patient. Importantly, the prosthetic valve is designed and configured to self-center within the target anatomy after deployment from the valve delivery system. The prosthetic valve is configured to self-center even with non-coaxial delivery or placement of the valve within the annulus and anchor. Coaxial delivery in this context refers to a central (longitudinal) axis of the prosthetic valve and a central axis of the anchor (e.g., axis perpendicular to the plane(s) containing the anchor). For example, the frame is tolerant to up to 45 degrees of off-axis delivery. The stiffness of the wide atrial brim enables this self-centering, balanced against the softness or compliance of the atrial flared portion to be atraumatic and prevent damage to tissue of the atrium and annulus. The short axial height of the ventricular flare or ventricular side of the prosthetic valve (e.g., less than 10 mm) allows the valve to deploy and self-center.

The prosthetic mitral valve of the present disclosure prevents paravalvular leaks (PVL) after implantation. The frame design, including the combination of a soft and wide atrial brim, a narrow central waist that interacts with the anchor to pinch inferior/superior to the annulus, and the fabric selection of the valve completely seals the valve against the anatomy reducing or eliminating the risk of blood flowing between the implanted valve and the cardiac tissue. Once the valve is implanted, is seated to the atrial floor with the wide atrial brim. The prosthetic valve of the present disclosure is further designed and configured to reduce or limit left ventricular outflow tract obstruction (LVOTO). The short ventricular height of the valve (e.g., less than 10 mm), the self-centering nature of the valve (e.g., optimizing the angle of the valve with respect to the LVOT), and the tissue interaction between the valve and the anatomy (e.g., anterior leaflet capture/superior adjustment) all provide for a solution to LVOTO that has not been achieved with other competing devices. Specifically, the valve and anchor capture and pull the anterior leaflet away from the LVOT during expansion of the valve and axial adjustment of the anchor position, further reducing LVOTO.

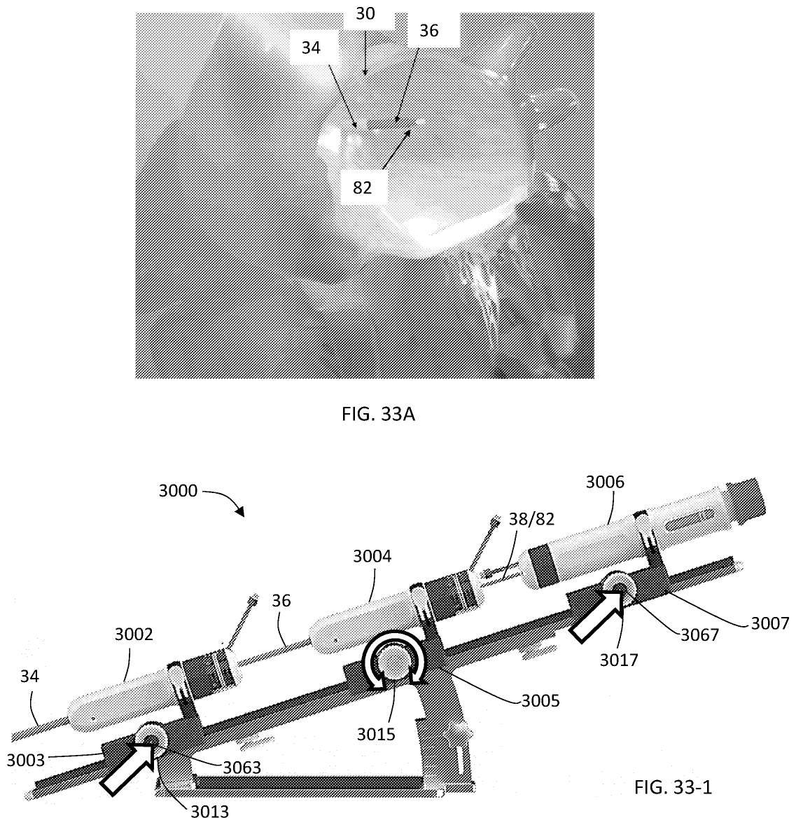

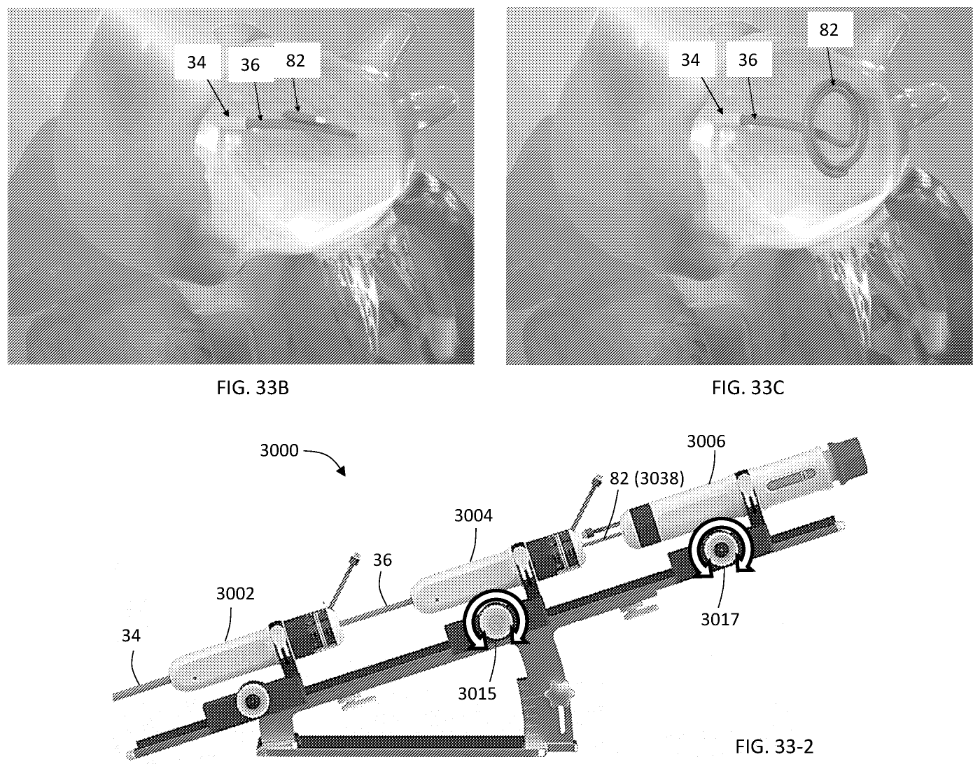

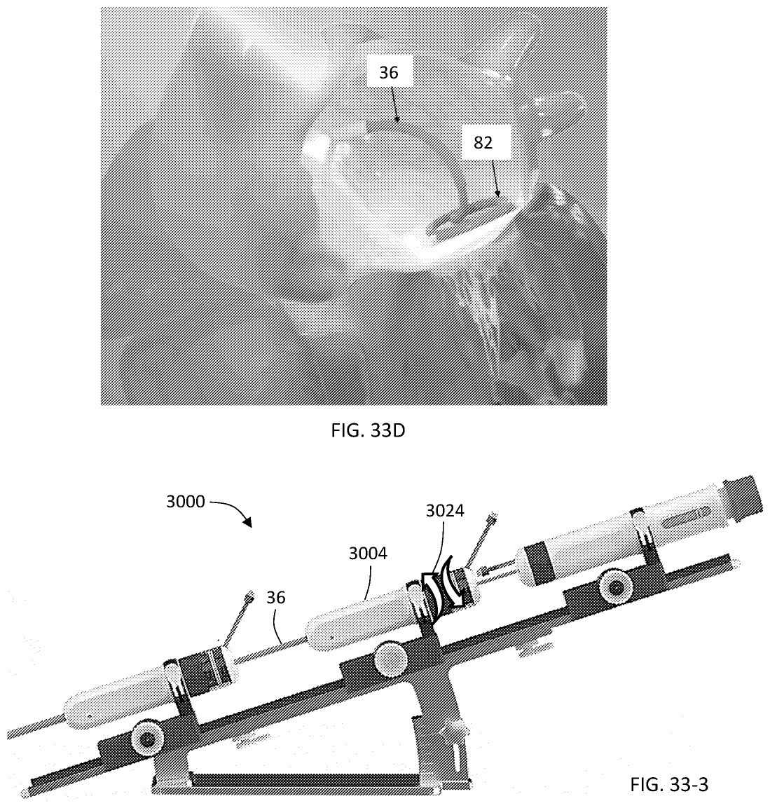

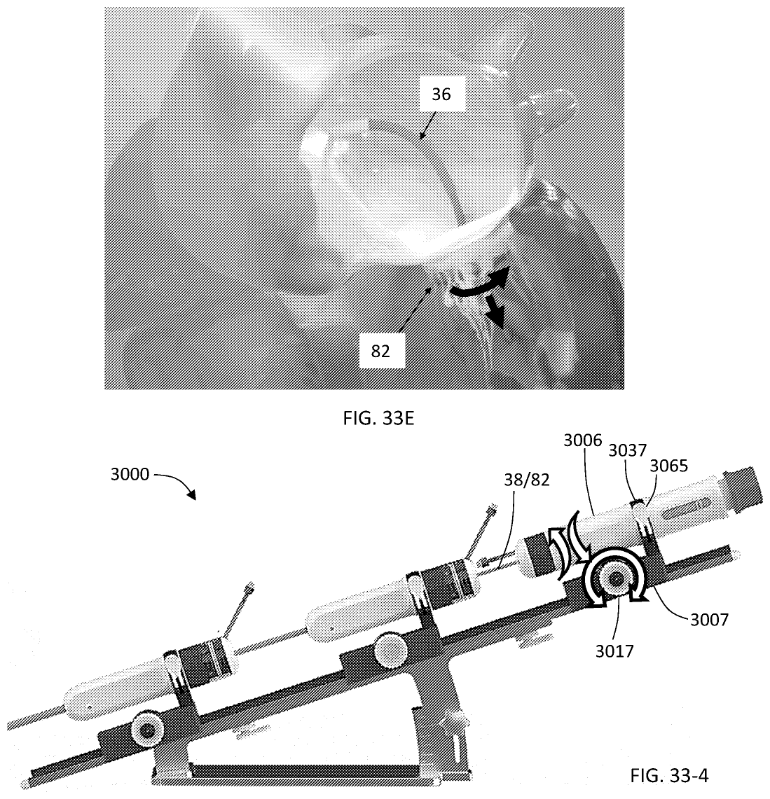

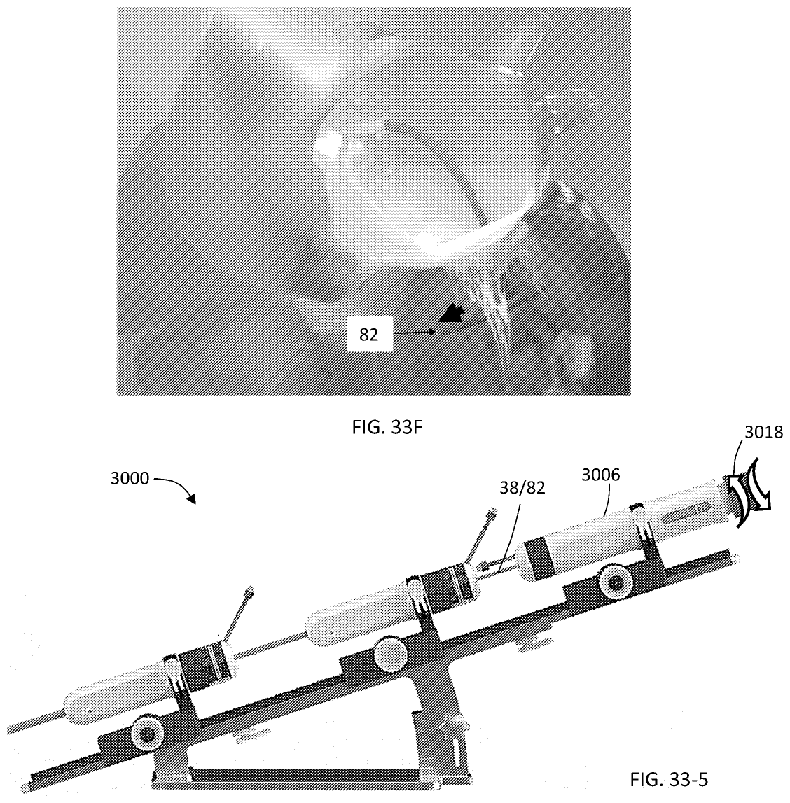

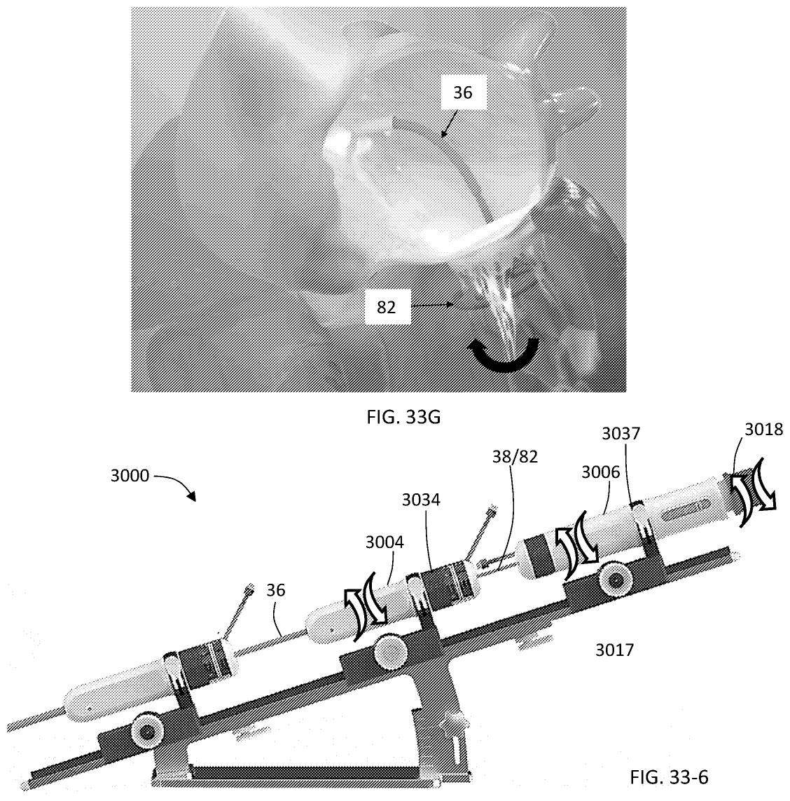

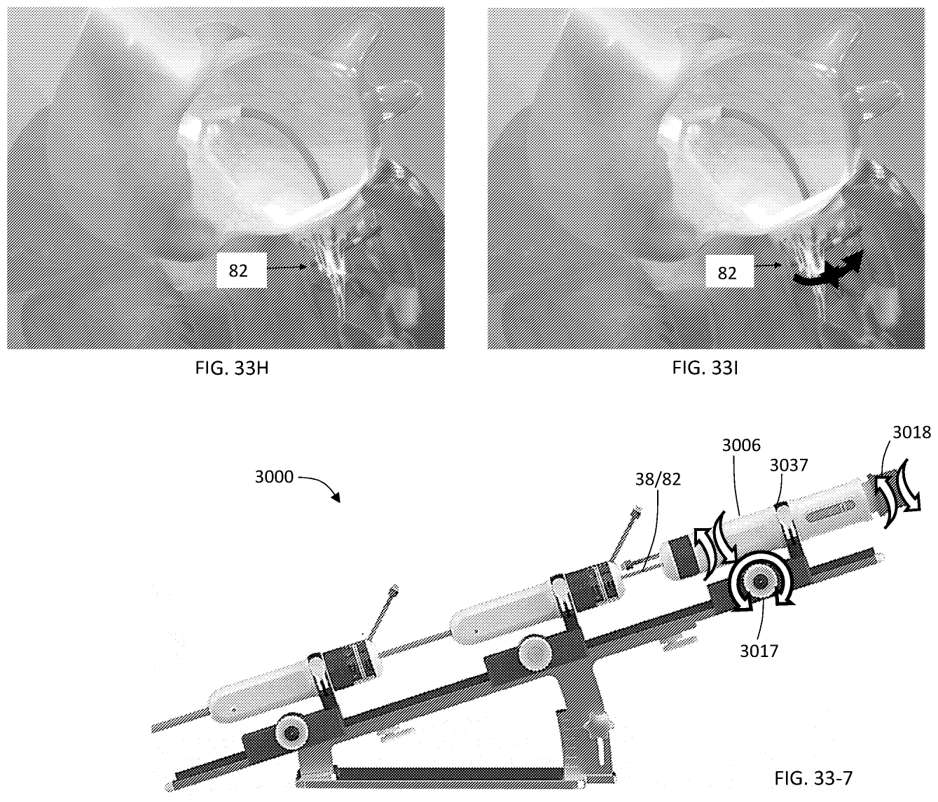

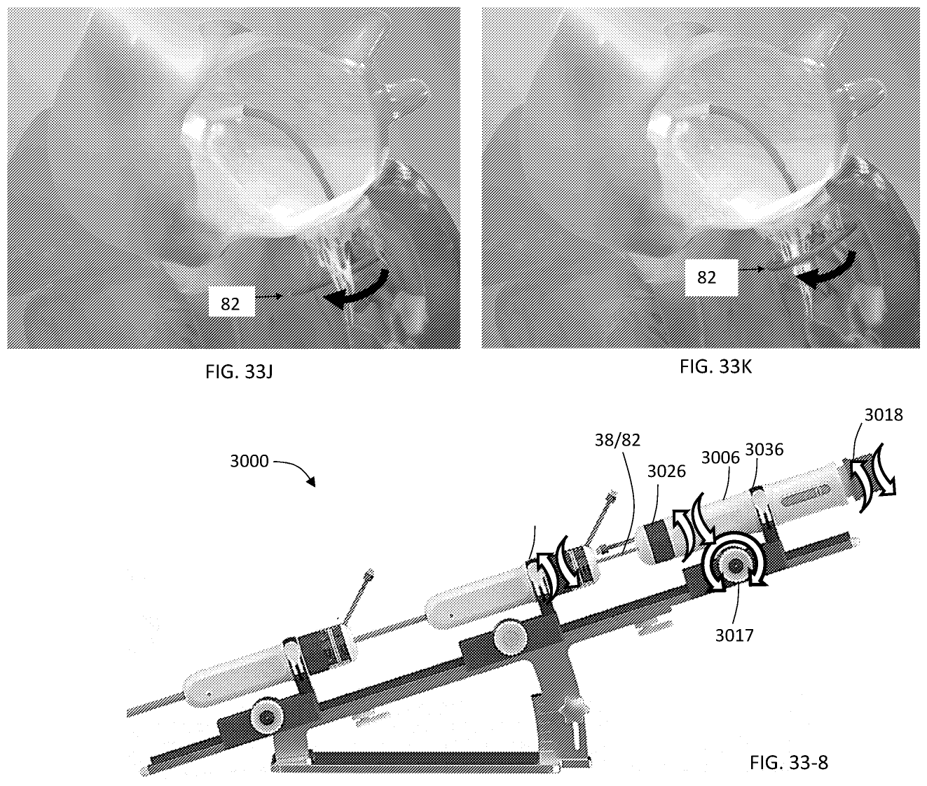

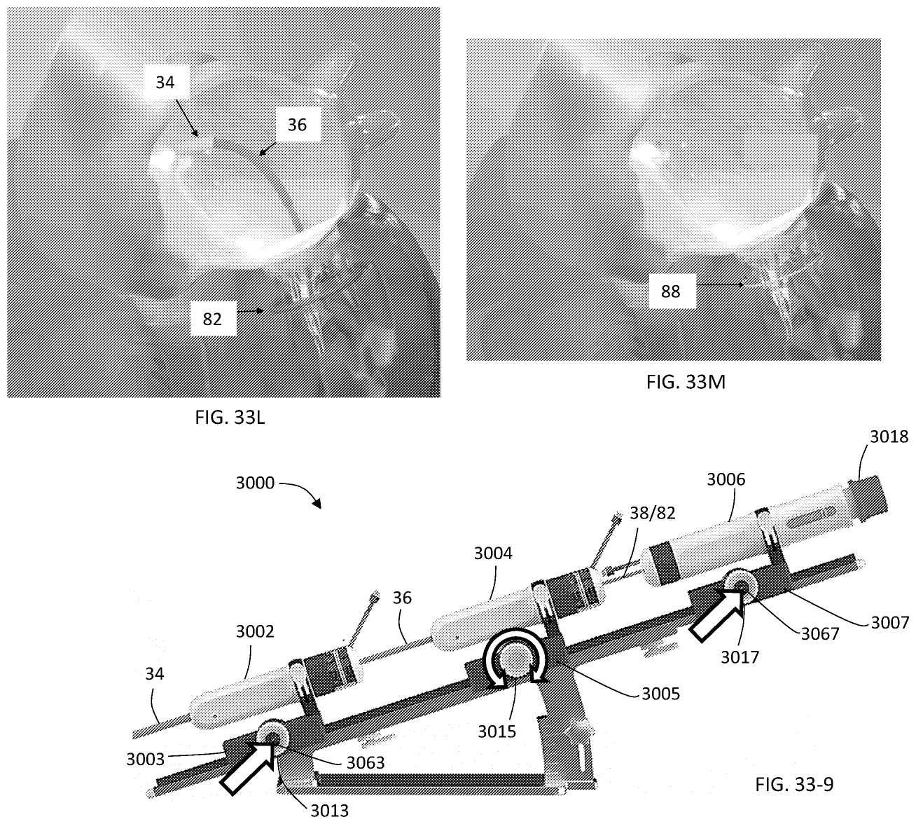

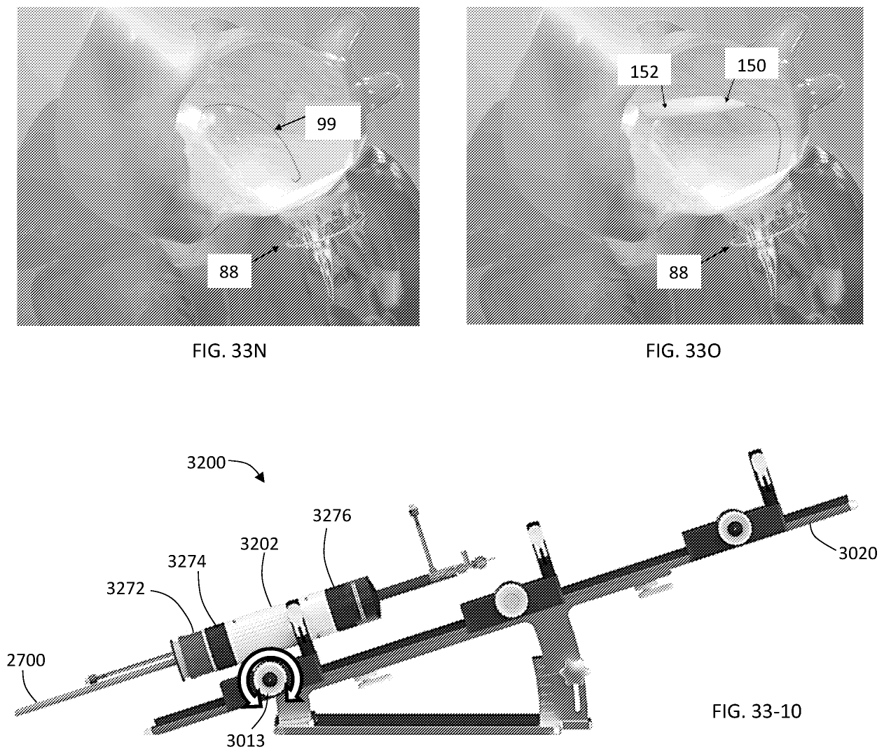

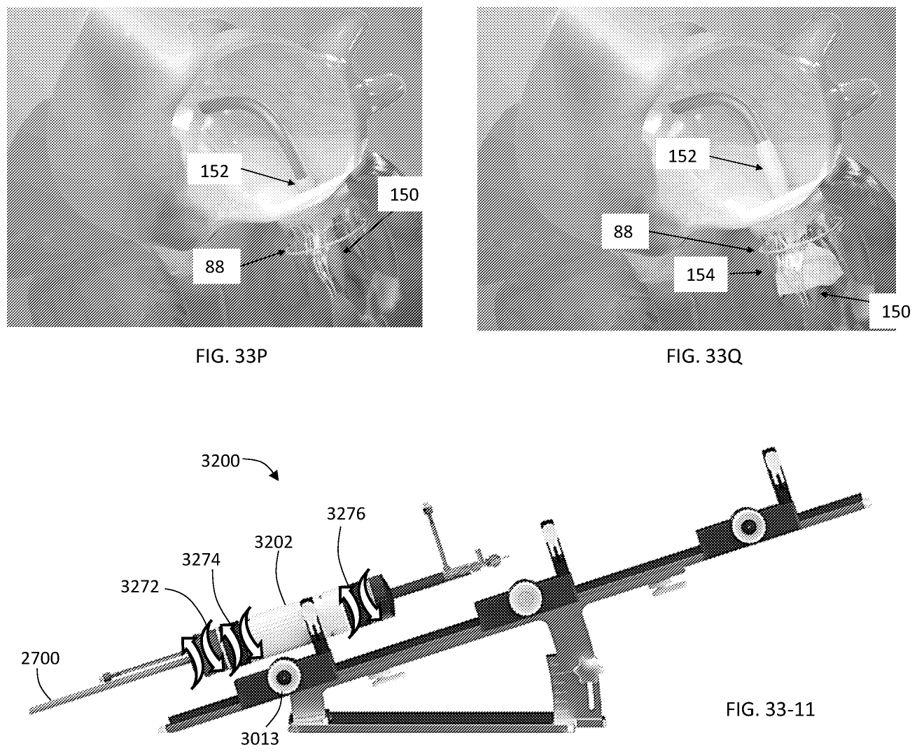

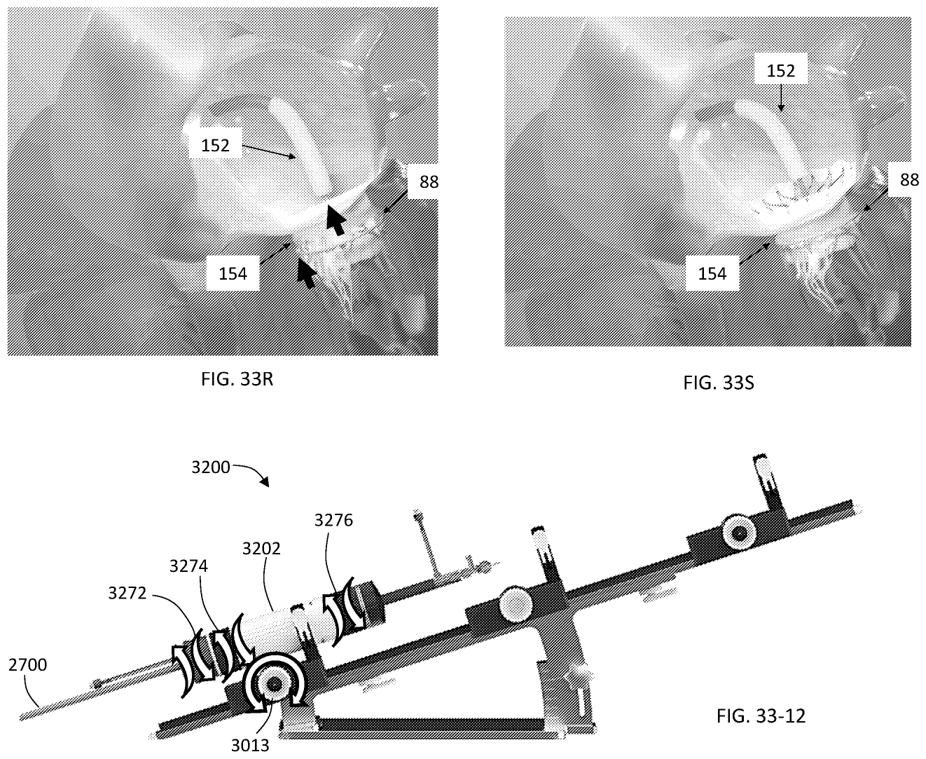

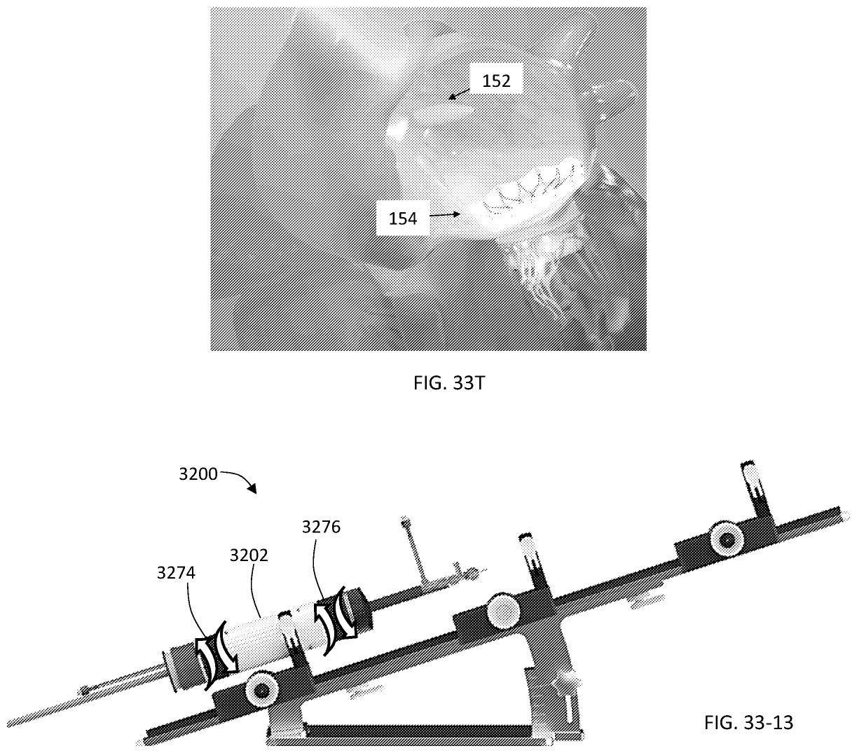

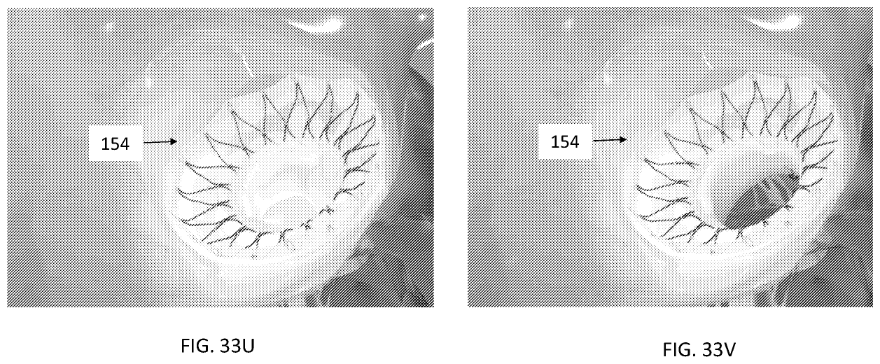

Referring to FIG. 33A, a nested catheter system of an anchor delivery subsystem, which includes an outer steerable catheter 34, an inner steerable catheter 36, and a guide arm 82, is navigated to the patient's right atrium and through the septum to the left atrium of the subject's heart. In some cases, the nested catheter system is advanced through the patient's vasculature by hand by the user (e.g., surgeon). In some examples, a separate puncture procedure is used to puncture the septum prior to advancing the nested catheter system through the septum.

FIG. 33-1 shows an example manipulation of the anchor delivery controller 3000 for advancing the outer steerable catheter 34, the inner steerable catheter 36, and the guide arm 82 (which is at the distal portion of the anchor control catheter 38) together though the septum, as shown in FIG. 33A. The button 3063 of the first dial 3013 and the button 3067 of the third dial 3017 may be activated to couple translational movement of the first carriage 3003 and the third carriage 3007 with translational movement of the second carriage 3005. The second knob 3015 may then be rotated to advance the outer steerable catheter 34, the inner steerable catheter 36, and the guide arm 82 together though the septum. The buttons 3063 and 3067 may then be deactivated.

FIGS. 33B and 33C show the inner steerable catheter 36 being advanced out of the distal end of the outer steerable catheter 34 into the left atrium. In addition, the guide arm 82 is advanced out of the distal end of the inner steerable catheter 36 into the left atrium. The active and passive portions of the guide arm 82, in combination with the anchor carried within, enable the guide arm 82 to self-assemble to form, in this case, a spiral shape (FIGS. 14A-14B) in a left atrium. As previously described, in other examples, the guide arm 82 is configured to self-assemble into a helical shape (FIGS. 15A-15B). As previously described, the anchor also fully self-assembles and assumes its at-rest shape within the guide arm 82 when the guide arm 82 is deployed in the left atrium.

FIG. 33-2 shows an example manipulation of the anchor delivery controller 3000 for advancing the inner steerable catheter 36 and the guide arm 82 as shown in FIGS. 33A and 33B. The user may rotate the second dial 3015 to advance the inner steerable catheter 36 distally relative to the outer steerable catheter 34. The user may rotate the third dial 3017 to advance the guide arm 82 distally relative to the inner steerable catheter 36 and the outer steerable catheter 34.

In FIG. 33D, the inner steerable catheter 36 is deflected to steer the guide arm 82 towards the mitral annulus. In the illustrated example, the spiral shape of the guide arm 82 (e.g., the portion of the guide arm 82 distal to the bend (93 in FIG. 14B) is generally parallel with the mitral annulus. In cases where the guide arm 82 has a helical shape, the portion of the guide arm 82 distal to the bend (93 in FIG. 15B) can have a helical shape.

FIG. 33-3 shows how the anchor delivery controller 3000 can be used to steer the guide arm 82 as shown in FIG. 33D. The user may rotate the rotation knob 3024 of the second handle 3004 to cause the inner steerable catheter 36 to deflect, thereby steering the guide arm 82 toward the mitral annulus, as shown in FIG. 33D.

In FIG. 33E, the guide arm 82 is counter-rotated and advanced to cross the mitral valve, through the leaflets, and into the left ventricle. In this example, the guide arm 82 is fully deployed and the anchor is fully assembled within the guide arm 82 when the guide arm 82 is advanced across the mitral valve. The shape of the guide arm 82 (and the planarity of the guide arm 82 with respect to the mitral valve) can be maintained as the guide arm 82 crosses the mitral valve, as shown.

FIG. 33-4 shows how the anchor delivery controller 3000 can be used to rotate the guide arm 82 as shown in FIG. 33E. The user may unlock (e.g., pull) a lever 3065 of the fastener 3037 of the third carriage 3007 to release tension on the band portion of the fastener 3037. The user may then rotate the handle 3006, thereby causing the anchor control catheter 38 to rotate in. Since the guide arm 82 is a distal part of the anchor control catheter 38, the guide arm 82 also rotates. To advance the guide arm 82, the user may rotate the dial 3017 (e.g., third dial) to advance the handle 3006 and the guide arm 82 distally.

Referring to FIG. 33F, the encircling process can begin. As described previously, the distal tip of the guide arm 82 can be extended radially outwards, for example by selected proximal retraction of the anchor therein. In the example illustrated, the distal tip of the guide arm 82 is extended toward the left ventricular outflow tract (LVOT).

FIG. 33-5 shows how the anchor delivery controller 3000 can be used to extend the distal tip of the guide arm 82 as illustrated in FIG. 33F. The user may rotate the proximal knob 3018 to advance and/or retract the anchor within the guide arm 82. As described previously with reference to FIGS. 18A-18C, movement of the anchor within the guide arm 82 can cause the distal part of the guide arm 82 to extend radially outward. In some examples, after the anchor is moved within the guide arm 82 to cause sufficient radial extension of the distal tip of the guide arm 82, the proximal knob 3018 may be locked to keep the guide arm 82 in the desired radially extended state.

In FIG. 33G, the guide arm 82, while in a radially extended state, is rotated to encircle chordae/leaflets within the left ventricle. Since the encircling process can be performed under echo imaging to provide visualization of the guide arm 82, the user can actively manipulate the distal tip of the guide arm 82 to capture the desired chordae/leaflets. This may include extending the distal tip of the guide arm 82 radially outwards or pulling the distal tip radially inwards, depending on the patient specific anatomy.

FIG. 33-6 shows how the anchor delivery controller 3000 can be used to rotate and manipulate the guide arm 82 as shown in FIG. 33G. The user may unlock the fastener 3037 to release tension around the handle 3006. Then the user may rotate the handle 3006, thereby causing the anchor control catheter 38 with the guide arm 82 to rotate in the same direction. In some examples, the second handle 3004 may also be rotated (after unlocking the fastener 3034) to rotate the inner steerable catheter 36 to increase the reach of the distal tip of the guide arm 83. To actively manipulate the distal tip of the guide arm 82, the user can rotate the proximal knob 3018 to move the anchor proximally within the guide arm 82. This causes the distal tip of the guide arm 82 to extend radially and pull inward radially, thereby giving the user control to capture chordae/leaflets in different regions near the mitral annulus.

In FIG. 33H, the position of the guide arm 82 has been assessed and a determination made that at least some chordae or leaflet tissue has not been correctly encircled. For repositioning the anchor delivery subsystem, the clinician can readily and independently adjust the axial height, circumferential (rotational) position, and/or radial reach of the distal end of the guide arm 82 to de-encircle/re-encircle, as often as desired. This delivery flexibility, along with the distinctive visualization characteristics of the anchor delivery catheter, provides the clinician with precise and repeatable control of encircling chordae and/or leaflets. At FIG. 33I, the guide arm 82 is retracted or counter-rotated to partially de-encircle some portion of previously encircled chordae.

FIG. 33-7 shows how the anchor delivery controller 3000 can be used to manipulate the guide arm 82 as shown in FIGS. 33H and 33I. After unlocking the fastener 3037, the user may rotate the handle 3006 to rotate the guide arm 82, causing the guide arm 82 to de-encircle. To adjust the axial height of the guide arm 82, the user may rotate the dial 3017 to move the guide arm 82 distally and/or proximally. To adjust the radial reach of the distal end of the guide arm 82, the user may rotate the proximal knob 3018 in clockwise and/or counterclockwise directions.

FIGS. 33J and 33K show the process of re-encircling the guide arm 82 after counter-rotating the guide arm 82. The re-encircling process is used to fully capture the desired anatomy (chordae/leaflets). Adjusting or changing the radial position of the distal tip of the guide arm 82 can optionally be done at any point during encircling (rotating) or de-encircling (counter-rotating).

FIG. 33-8 shows how the anchor delivery controller 3000 can be used to manipulate the guide arm 82 as shown in FIGS. 33J and 33K. The user may rotate the handle to cause the guide arm 82 to rotate and re-encircle the chordae and/or leaflets. To adjust the radial position of the distal end of the guide arm 82, the user may rotate the proximal knob 3018.

Once the chordae are determined to be satisfactorily (e.g., fully) encircled by the guide arm 82, as shown in FIG. 33L, the guide arm 82 and the anchor delivery catheter system can be proximally retracted from the anchor without disturbing the anchor's position until the anchor delivery catheter system is removed from the patient. This is accomplished by maintaining the anchor and tether positions while withdrawing the guide arm 82 into the inner steerable catheter 36, until the distal end of the guide arm 82 clears the proximal end of the anchor and the anchor delivery catheter system is decoupled from the anchor. The inner steerable catheter 36 is then pulled into the outer steerable catheter 34 and the entire subsystem is removed from the subject. After the guide arm 82 is fully retracted and removed from the patient, the anchor 88 remains in place within the left ventricle surrounding the desired chordae/leaflets as shown in FIG. 33M. The anchor 88 may then be disconnected from the tether. The result is the anchor 88 is deployed and anchored around chordae and/or leaflets of the left ventricle with no connection to any other system component (e.g., no tether or other linkage is left behind when the anchor 88 is deployed). Additionally, while the anchor 88 is deployed around the chordae and/or leaflets, the axial position of the anchor 88 is not fixed to the anatomy. Therefore, the anchor 88 can slide or be moved axially (e.g., towards or away from the annulus). This is described more fully below when self-expansion and positioning of the valve is described.

FIG. 33-9 shows how the anchor delivery controller 3000 can be used to retract the guide arm 82 from the anchor 88 as shown in FIGS. 33L and 33M. The user may lock (or hold) the proximal knob 3018 to maintain the anchor position in place around the desired chordae/leaflets. While the proximal knob 3018 is locked (or held), the user may rotate the dial 3017 (e.g., third dial) to withdraw the guide arm 82 proximally into the inner steerable catheter 36. Retraction of the guide arm 82 over the anchor 88 causes the anchor to detach from the tether, as described herein. After the guide arm 82 is fully retracted, the user may rotate the dial 3015 (e.g., second dial) to withdraw the inner steerable catheter 36 proximally into the outer steerable catheter 34. After the inner steerable catheter 36 is retracted into the outer steerable catheter 34, the user may activate the button 3063 of the first dial 3013 and the button 3067 of the third dial 3007. This couples translational movement of the second carriage 3005 with the first carriage 3003 and the third carriage 3007. The second dial 315 may then be rotated to retract the catheters 34, 36 and 38 together out of the atrium.

At FIG. 33N, the valve delivery can be initiated. First, a guidewire 99 can be inserted through the septum, into the left atrium, through the mitral valve annulus, and through the anchor 88 into the left ventricle. In FIG. 33O, the valve delivery subsystem is advanced over the guidewire 99, through the septum, and into the left atrium. FIG. 33O shows the valve capsule 152 (containing the valve prosthesis) and the nose cone 150 in the left atrium.

FIG. 33-10 shows how the valve delivery controller 3200 can be used in the operations shown in FIGS. 33N and 330. At first, the valve delivery subsystem may be delivered over the guidewire 99 through the patient's vasculature while the valve delivery subsystem is not connected to the rail system. After the distal end of the valve delivery catheter is advance to approximately in the inferior vena cava (IVC) (e.g., before entering the heart chambers/crossing the septum), the valve delivery controller 3200 (which is coupled to the proximal end of the valve delivery subsystem) may be attached to the rail system 3020. The dial 3013 may be rotated to advance the valve capsule 152 and nose cone 150 in the left atrium as shown in FIG. 33O.

FIG. 33P shows the valve capsule 152 and nose cone 150 advanced through the annulus with the nose cone 150 positioned past the anchor 88 and the valve capsule 152 extending across/through the anchor 88. In FIG. 33Q, the valve capsule 152 is partially retracted from the nose cone 150, to allow for (partial) self-expansion or assembly of the ventricular portion of the valve prosthesis 154. In some embodiments, the ventricular portion of the valve 154 expands into the anchor 88.

FIG. 33-11 shows how the valve delivery controller 3200 can be used to advance the valve capsule 152 and partially release the valve prosthesis 154 as shown in FIGS. 33P and 33Q. The steering/flex shape control knob 3276 of the handle 3202 can be rotated to change a shape of the distal portion of the inner steerable catheter 2700, as shown in FIG. 33P. In addition, the depth control knob 3274 of the handle 3202 can be rotated to advance the valve capsule 152 and the nose cone 150, into the mitral annulus, as shown in FIG. 33P. The valve deployment knob 3272 can be rotated to retract the valve capsule 152 and to cause partial release of the valve prosthesis 154 into the left ventricle, as shown in FIG. 33Q.

In other embodiments, referring to FIG. 33R, the valve delivery subsystem can be retracted or pulled towards the mitral annulus to capture the ventricular portion of the frame of the valve 154 within the anchor 88. In some embodiments, the anchor 88 can be lifted or pulled towards the annulus with the partially expanded valve 154 by manipulation (e.g., proximal retraction) via the valve delivery subsystem, as indicated by the arrows. As described above, implanting the valve and anchor higher in the anatomy can help to reduce or prevent LVOTO. When the anchor 88 is lifted or pulled towards annulus, the anterior leaflet can be captured and bunched up by the anchor 88 into or against the annulus. This act of capturing the anterior leaflet, and pulling or bunching up the leaflet against the annulus moves tissues away from the LVOT, thereby reducing LVOTO.

In FIGS. 33S, the valve capsule 152 is fully retracted from the valve prosthesis 154 to expose the atrial portion and atrial brim 105 of the valve frame structure, allowing for self-expansion or the atrial portion of the valve prosthesis 154 within the left atrium. Once the atrial portion is fully expanded, the mitral annulus is sealed with the wide and conformable atrial brim 105 of the valve frame, the position of the atrial brim 105 maintained and/or pressed downward by the anchor firmly positioned at the valve 154 waist and capturing native tissues therebetween.

FIG. 33-12 shows how the valve delivery controller 3200 can be used to retract the catheter 2700 and to cause release of the atrial brim 105 of the valve 154, as shown in FIGS. 33R and 33S. Pulling the valve 154 proximally as shown in FIG. 33R may be accomplished by rotating the depth control knob 3274 and/or the dial 3013. The valve deployment knob 3272 can be rotated to retract the valve capsule 152 over the valve 154 and release the remainder of the valve 154 from the valve capsule 152.

In FIG. 33T, the valve delivery subsystem is being removed, leaving the valve prosthesis 154 implanted within the native mitral valve and the anchor 88, and thereby sealing the native mitral annulus.

FIG. 33-13 shows how the valve delivery controller 3200 can be used to retract the catheter 2700 to cause retraction of the valve delivery subsystem from the native mitral valve, as shown in FIG. 33T. The depth control knob 3274 of the handle 3202 can be rotated to retract the catheter 2700, including the valve capsule 152, from the patient's heart and body. During retraction process, the steering/flex shape control knob 3276 of the handle 3202 may be rotated to straighten the distal portion of the catheter 2700 from the deflected state.

FIGS. 33U and 33V illustrate a left-atrial view of the valve frame prosthesis 154 implanted within the mitral annulus with the leaflets closed (FIG. 33U) and opened (FIG. 33U).

The valve delivery subsystem is a low profile valve delivery system that allows control of valve position until the very end of the delivery procedure. The valve delivery subsystem is a true 28Fr delivery profile with steerability that allows for familiar and easy positioning of the valve frame in the target location. The valve delivery subsystem allows for deployment of the collapsed or compressed valve frame within an already deployed anchor. Expansion of the valve frame structure captures the anchor and controls the final anchor position. Emphasis has been provided above that it is desirable to have the anchor deployed in a “high” position towards the left atrium so as to avoid LVOTO. While the anchor position can be controlled with the anchor delivery subsystem, it should also be understood that the anchor position can be adjusted or pulled upwards with the valve delivery subsystem after the valve has been allowed to expand within the anchor.

This disclosure provides details around a forgiving mitral valve replacement procedure and system specifically designed for the mitral anatomy. The systems and methods disclosed herein solve for an unmet need by providing a delivery system and delivery procedure that is familiar to physicians with a small learning curve, an implant that is adaptable and applicable to all anatomies, and an implant that reliable eliminates mitral regurgitation (MR) without the risk of complications associated with other mitral valve replacement devices on the market.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

A complete listing of the claims follows:

1. A delivery system for a prosthetic heart valve, the prosthetic heart valve comprising an anchor adapted to be disposed in a ventricle adjacent a native valve of a patient's heart and a frame supporting valve leaflets adapted to be expanded within the anchor, the delivery system comprising:
an anchor control catheter adapted to be advanced into an atrium of the patient's heart, the anchor control catheter comprising:
a lumen extending from a proximal end to a distal end of the anchor control catheter, the lumen being sized and configured to slidingly contain the anchor;
a distal guide arm in a distal portion of the anchor control catheter, at least a portion of the distal guide arm having an at-rest spiral shape; and
a proximal controller at the proximal end of the anchor control catheter, the proximal controller being configured to change a shape of the distal guide arm.

2. The delivery system of claim 1, wherein the distal guide arm has a proximal portion and a distal portion, the proximal portion comprising the portion of the distal guide arm having an at-rest helical or spiral shape.

3. The delivery system of claim 2, wherein the proximal controller comprises an actuator operatively connected to the anchor in the lumen of the anchor control catheter to move the anchor distally and proximally within the lumen to change the shape of the distal portion of the distal guide arm.

4. The delivery system of claim 3, wherein the actuator is connected to a tether which is removably connected to the anchor.

5. The delivery system of claim 2, wherein the proximal controller comprises an actuator operatively connected to the distal portion of the distal guide arm and adapted to change a shape of the distal portion of the distal guide arm.

6. The delivery system of claim 5, wherein the actuator is connected to an actuation catheter movably disposed within the lumen of the anchor control catheter, a distal end of the actuation catheter being connected to the distal portion of the distal guide arm.

7. The delivery system of claim 2, wherein the proximal controller comprises an actuator operatively connected to a proximal portion of the anchor control catheter and adapted to rotate the anchor control catheter.

8. The delivery system of claim 1, wherein the distal guide arm is sized and configured to move to a helical or spiral shape within the atrium of the patient's heart.

9. The delivery system of claim 8, wherein the proximal controller is further configured to extend the distal guide arm from the atrium through valve leaflets into the ventricle with the anchor disposed within the lumen.

10. The delivery system of claim 9, wherein the proximal controller is further configured to move a distal end of the distal guide arm within the ventricle to encircle chordae of the heart with the distal guide arm.

11. The delivery system of claim 10, wherein the proximal controller is further configured to withdraw the anchor control catheter from the anchor after the distal guide arm has encircled the chordae.

12. A delivery system for a prosthetic heart valve, the prosthetic heart valve comprising an anchor adapted to be disposed in a ventricle adjacent a native valve of a patient's heart and a frame supporting valve leaflets adapted to be expanded within the anchor, the delivery system comprising:
a valve capsule, the valve frame being disposed within the valve capsule in a compressed configuration;
a capsule shaft catheter connected to the valve capsule and extending proximally from the valve capsule;
a valve retainer removably connected to the valve frame; and
a proximal controller at a proximal end of the capsule shaft catheter, the proximal controller being configured to remove the capsule from the valve frame, thereby permitting the valve frame to expand.

13. The delivery system of claim 12, further comprising an inner steerable catheter disposed within a lumen of the capsule shaft catheter and an inner catheter steering control line extending from a distal portion of the inner steerable catheter to the proximal controller, the proximal controller being further configured to apply and release tension on the inner catheter steering control line.

14. The delivery system of claim 13, further comprising an outer steerable catheter and an outer catheter control line extending from a distal portion of the outer steerable catheter to the proximal controller, the proximal controller being further configured to apply and release tension on the outer catheter control line, the capsule shaft catheter being disposed in a lumen of the outer steerable catheter.

15. The delivery system of claim 13, wherein the capsule shaft catheter includes multiple axial sections having different stiffnesses, thereby providing different degrees of deflection when activated.

16. The delivery system of claim 15, wherein, when the capsule shaft catheter is in a deflected state, wherein the capsule shaft catheter includes a first bend and a second.

17. The delivery system of claim 16, wherein the first bend is configured to be in a right atrium of the patient's heart and the second bend is configured to be within a left atrium of the patient's heart.

18. A track system adapted to control movement of a catheter system for delivering at least a portion of a prosthetic heart valve into a patient's heart, wherein the catheter system includes a first catheter coaxially arranged with a second catheter, the track system comprising:
a primary track and a secondary track positioned in parallel;
a first carriage adapted to secure a proximal portion of the first catheter thereto and to translate along the primary track, wherein the first carriage is coupled to the secondary track such that the secondary track translates with the first carriage when the first carriage translates along the primary track; and
a second carriage adapted to secure a proximal portion of the second catheter thereto and to translate along the primary track, wherein the second carriage includes a coupler that is adapted to selectively engage the second carriage with the secondary track such that, when the coupler is engaged, the second carriage translates with the first carriage when the first carriage translates along the primary track.

19. The track system of claim 18, wherein the first carriage includes a fastener that is configured to transition between:
a first closed state in which the proximal portion of the first catheter is frictionally secured to the first carriage, wherein the first catheter is maintained at an intended rotational position but is rotatable with respect to the first carriage; and
a second closed state in which the proximal portion of the first catheter is fully secured to and not rotatable with respect to the first carriage.

20. The track system of claim 18, further comprising a third carriage adapted to secure a proximal portion of a third catheter thereto and to translate along the primary track, wherein the third carriage includes a second coupler that is adapted to selectively engage the third carriage with the secondary track such that, when the second coupler is engaged, the third carriage translates with the first carriage when the first carriage translates along the primary track.

21.-73. (canceled)
